SEQUENCE LISTING (1 sequence)

SEQ ID NO: 1 - DNA, 20 bp, Artificial Sequence
  antisense oligonucleotide
ctctagcgtcttaaagccga

(19) United States
(12) Patent Application Publication
(10) Pub. No.: US 2007/0274947 A1
(43) Pub. Date: Nov. 29, 2007

(54) Antisense Oligonucleotides Directed to Ribonucleotide Reductase R1 and Uses Thereof in the Treatment of Cancer

(76) Inventors: Aiping H. Young, Toronto (CA); Jim A. Wright, Oakville (CA)

Correspondence Address: Ivor R. Elrifi, Ph.D.;Mintz, Levin, Cohn, Ferris, Glovsky and Popeo, PC, One Financial Center, Boston, MA, 02111, US

(21) Appl. No.: 10/557,853

(22) PCT Filed: May 21, 2004
(86) PCT No.: PCT/CA04/00761

§ 371 Date: Jan. 29, 2007

Related U.S. Application Data
(60) Provisional application No. 60/320,210, filed on May 21, 2003.

(51) Int. Cl. A61K 31/7052; A61K 31/7056; A61K 38/20; A61P 35/00; A61P 35/02

(52) U.S. Cl. 424/85.2; 514/44; 514/49

(57) ABSTRACT

The present invention provides antisense oligonucleotides directed to a mammalian ribonucleotide reductase R1 gene and combinations of the antisense oligonucleotides with one or more chemotherapeutic agents for use in the treatment of cancer.

### REFERENCE TO RELATED APPLICATIONS

This application is a Continuation-In-Part of U.S. patent application Ser. No. 10/447,136 filed May 29, 2003, which is a Continuation of U.S. patent application Ser. No. 09/249,247 filed Feb. 11, 1999, which is a Continuation-In-Part of U.S. patent application Ser. No. 08/904,901 filed Aug. 1, 1997, which in turn claims priority to U.S. Provisional Application No. 60/023,040 filed Aug. 2, 1996 and U.S. Provisional Application No. 60/039,959 filed Mar. 7, 1997, each of which is incorporated herein by reference in its entirety.

### FIELD OF THE INVENTION

The present invention pertains to the field of cancer therapeutics and in particular to the use of antisense oligonucleotides alone or in combination with one or more chemotherapeutic drugs for the treatment of cancer.

### BACKGROUND

The first unique step leading to DNA synthesis is the conversion of ribonucleotides to their corresponding deoxyribonucleotides, a reaction that is catalyzed in a cell cycle specific manner by the housekeeping gene ribonucleotide reductase [Lewis et al., 1978; Reichard, 1993; Wright, 1989a; Wright et al., 1990a; Stubbe, 1989]. The mammalian enzyme is composed of two dissimilar dimeric protein components often called R1 and R2, which are encoded by two different genes located on different chromosomes [Bjorklund et al., 1993; Tonin et al., 1987].

The levels of the R1 protein do not appear to change substantially during the cell cycle of proliferating cells and can be detected throughout the cell cycle. Synthesis of R1 mRNA, like R2 mRNA appears to occur mainly during S phase [Eriksson et al., 1984; Choy et al., 1988; Mann et al., 1988]. The broader distribution of the R1 protein during the cell cycle is attributed to its longer half life as compared to the R2 protein [Choy et al., 1988; Mann et al., 1988].

Regulation of ribonucleotide reductase, and particularly the R2 component, is altered in malignant cells exposed to some tumour promoters and to the growth factor TGF-β [Amara, et al., 1994; Chen et al., 1993; Amara et al., 1995b; Hurta and Wright, 1995; Hurta et al., 1991]. Higher levels of enzyme activity have been observed in cultured malignant cells when compared to nonmalignant cells [Weber, 1983; Takeda and Weber, 1981; Wright et al., 1989a], and increased levels of R2 protein and R2 mRNA have been found in pre-malignant and malignant tissues as compared to normal control tissue samples [Saeki et al., 1995; Jensen et al., 1994]. However, these correlative studies did not show a direct role for ribonucleotide reductase in cancer cell transformation and tumor progression, because like so many other enzyme activities found to be altered in cancer cells [e.g. Weber, 1983], the results could easily be explained by the increased cell proliferation and altered cell cycle regulation characteristics of transformed and malignant cell populations [Morgan and Kastan, 1997].

Antisense oligonucleotides directed to the R1 or R2 component of ribonucleotide reductase have been shown to be effective in reducing the growth of cancer cells [see, for example, U.S. Pat. Nos. 5,998,383 and 6,121,000].

In view of the high incidence of various types of cancer throughout the world, there remains a need for improved therapies for the treatment of cancer.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide antisense oligonucleotides directed to ribonucleotide reductase R1 and uses thereof in the treatment of cancer. In accordance with an aspect of the present invention, there is provided an antisense oligonucleotide of between 7 and 100 nucleotides in length comprising at least 7 consecutive nucleotides from SEQ ID NO:1 for use in the treatment of cancer in a mammal in need of such therapy.

In accordance with another aspect of the present invention, there is provided an antisense oligonucleotide of between 7 and 100 nucleotides in length comprising at least 7 consecutive nucleotides from SEQ ID NO:1 for use in combination with one or more chemotherapeutic agents in the treatment of cancer in a mammal in need of such therapy.

In accordance with another aspect of the present invention, there is provided an antisense oligonucleotide of between 20 and 100 nucleotides in length comprising the sequence as set forth in SEQ ID NO:1 for use in combination with one or more chemotherapeutic agents in the treatment of a human having a cancer selected from the group of: a solid tumour, lymphoma, renal cancer, breast cancer, lung cancer, prostate cancer, ovarian cancer, cervical cancer, colon cancer and leukaemia.

In accordance with another aspect of the present invention, there is provided a use of an antisense oligonucleotide of between 7 and 100 nucleotides in length comprising at least 7 consecutive nucleotides from SEQ ID NO:1 in the manufacture of a medicament for the treatment of cancer.

### BRIEF DESCRIPTION OF THE FIGURES

FIG. 1 depicts the effects of the nucleotide sequence according to SEQ ID NO:1 on PC-3 and DU145 Prostate Tumor Growth in SCID Mice. Treatment with SEQ ID NO:1 demonstrated a strong inhibitory effect on the growth of human prostate carcinoma.

FIG. 2 depicts the effects of the nucleotide sequence according to SEQ ID NO:1 on DU145 Prostate Tumor Growth in SCID Mice. The anti-tumor effect of SEQ ID NO:1 was further compared to that of mitoxantrone (novantrone®) alone or in combination (A and B).

FIG. 3 depicts the effects of anti-tumor activity of SEQ ID NO:1 on Caki-1 Human Kidney Tumor Growth in SCID/beige mice that are NK, T and B cell deficient; A) Tumor Size and B) Tumor Weight.

FIG. 4 depicts the effects of SEQ ID NO:1 on R1 mRNA levels in HT-29 colon tumors in CD1 nude mice having HT-29 xenografts.

FIG. 5 depicts measurements of R1 protein levels using Western blot analysis and AD 203, an anti-R1-antibody, in untreated cancer cell lines derived from diverse human cancer types, including renal (Caki 1 and A498), skin (A2058), colon (HT-29) and breast (MDS-MB-231) cancer cell lines. The R1 protein expression was compared to R1 expression in 2 normal cell lines, WI38 and HUVEC.

FIG. 6 depicts the effect of SEQ ID NO:1 on the colony forming ability in the human tumor cell lines, Hep G2 (liver), SK-OV-3 (ovary), U-87 MG (brain), A2058 (melanoma), H460 (lung), MDA-MB-231 (breast) and AsPC-1 (pancreas).

FIG. 7 depicts a Northern blot analysis of the effect of SEQ ID NO:1 on R1 mRNA levels in the human tumor cell lines HT-29 (human colon adenocarcinoma) and MDA-MB-231 (human breast adenocarcinoma) cell lines.

FIG. 8 depicts the effect of SEQ ID NO:1 on the inhibition of the R1 target at the protein level in AsPC-1 human tumor cells (pancreatic adenocarcinoma) using immunoprecipitation analyses.

FIG. 9 depicts the effect of SEQ ID NO:1 on the inhibition of the R1 target at the protein level in MDA-MB-231 human breast adenocarcinoma using immunoprecipitation analyses.

FIG. 10 depicts a northern blot analyses of other cellular RNA levels in A2058 human melanoma cells treated with SEQ ID NO:1 or a scrambled control analogue of SEQ ID NO:1 in order to examine the specificity of inhibition of R1 mRNA by SEQ ID NO:1.

FIG. 11 depicts the effects of the nucleotide sequence according to SEQ ID NO:1 on SIHA human cervical carcinoma cell growth in SCID mice; A) Tumor Size and B) Tumor Weight.

FIG. 12 depicts the effects of the nucleotide sequence according to SEQ ID NO:1 on C8161 human melanoma cell lung nodule formation in experimental metastasis assays A) Ex vivo; and B) In vivo.

FIG. 13 depicts the effects of the nucleotide sequence according to SEQ ID NO:1 on HT-29 human colon tumor growth in CD-1 nude mice compared to A) mitomycin C alone or in combination; and B) CPT-11 alone or in combination.

FIG. 14 depicts the effects of the nucleotide sequence according to SEQ ID NO:1 on MDA231/CDDPs4 human cisplatin-resistant breast tumor growth in CB-17 SCID mice alone and in combination with taxol; A, B, D) Tumor Weight and C) Tumor Size.

FIG. 15 depicts the effects of the nucleotide sequence according to SEQ ID NO:1 on MDA-MB435-To.1 human breast adenocarcinoma resistant to taxol tumor growth in SCID mice alone and in combination with cisplatin; A and C) Tumor Weight; and B) Tumor Size.

FIG. 16 depicts the effects of the nucleotide sequence according to SEQ ID NO:1 on LS513 human multi-drug resistant colon adenocarcinoma tumor growth in SCID mice alone or in combination with CPIT-11; A) Tumor Size; and B and C) Tumor weight.

FIG. 17 depicts the effects of the nucleotide sequence according to SEQ ID NO:1 on BL-60 human promyelocytic leukemia growth in SCID mice; A) Tumor Size; and B) Tumor weight.

FIG. 18 depicts the effects of the nucleotide sequence according to SEQ ID NO:1 on survival time of SCID mice bearing Raji human Burkitt's lymphoma; A) and B) comparison with scrambled control SEQ ID NO:1-SCR.

FIG. 19 depicts the effects of the nucleotide sequence according to SEQ ID NO:1 on survival time of CB-17 SCID mice bearing mouse erythroleukemia (CB7).

### DETAILED DESCRIPTION OF THE INVENTION

The present invention relates to antisense oligonucleotides against the gene encoding a mammalian ribonucleotide reductase R1 protein and combinations of such antisense oligonucleotides and one or more chemotherapeutic agents in the treatment of various cancers. The antisense oligonucleotides and combinations of antisense oligonucleotides with one or more chemotherapeutic agents are more effective in decreasing the growth and/or metastasis of cancers, than treatment with the antisense oligonucleotide or the chemotherapeutic agent(s) alone. In one embodiment, the cancers are refractory cancers. In another embodiment the cancers are advanced cancers. In another embodiment the cancers are drug resistant cancers.

Definitions

Unless defined otherwise, all technical and scientific terms used herein have the same meaning as commonly understood by one of ordinary skill in the art to which this invention pertains.

The term “antisense oligonucleotide” as used herein means a nucleotide sequence that is complementary to the mRNA for a desired gene. In the context of the present invention, the desired gene is the gene encoding a mammalian ribonucleotide redustase R1 protein.

The term “selectively hybridise” as used herein refers to the ability of a nucleic acid to bind detectably and specifically to a second nucleic acid. Oligonucleotides selectively hybridise to target nucleic acid strands under hybridisation and wash conditions that minimise appreciable amounts of detectable binding to non-specific nucleic acids. High stringency conditions can be used to achieve selective hybridisation conditions as known in the art and discussed herein.

Typically, hybridisation and washing conditions are performed at high stringency according to conventional hybridisation procedures. Washing conditions are typically 1-3×SSC, 0.1-1% SDS, 50-70° C. with a change of wash solution after about 5-30 minutes.

The term “corresponds to” as used herein with reference to nucleic acid sequences means a polynucleotide sequence that is identical to all or a portion of a reference polynucleotide sequence. In contradistinction, the term “complementary to” is used herein to mean that the polynucleotide sequence is identical to all or a portion of the complement of a reference polynucleotide sequence. For illustration, the nucleotide sequence “TATAC” corresponds to a reference sequence “TATAC” and is complementary to a reference sequence “GTATA”.

The following terms are used herein to describe the sequence relationships between two or more polynucleotides: “reference sequence,” “window of comparison,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA or gene sequence, or may comprise a complete cDNA or gene sequence. Generally, a reference polynucleotide sequence is at least 20 nucleotides in length, and often at least 50 nucleotides in length.

A “window of comparison”, as used herein, refers to a conceptual segment of the reference sequence of at least 15 contiguous nucleotide positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A) (1988) 85:2444), using computerised implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e. resulting in the highest percentage of identity over the comparison window) is then selected.

The term “sequence identity” means that two polynucleotide sequences are identical (i.e. on a nucleotide-by-nucleotide basis) over the window of comparison.

The term “percent (%) sequence identity,” as used herein with respect to a reference sequence is defined as the percentage of nucleotide residues in a candidate sequence that are identical with the residues in the reference polynucleotide sequence over the window of comparison after optimal alignment of the sequences and introducing gaps, if necessary, to achieve the maximum percent sequence identity, without considering any conservative substitutions as part of the sequence identity.

The term “substantial identity” as used herein denotes a characteristic of a polynucleotide sequence, wherein the polynucleotide comprises a sequence that has at least 50% sequence identity as compared to a reference sequence over the window of comparison. Polynucleotide sequences at least 60% sequence identity, at least 70% sequence identity, at least 80% sequence identity, and at least 90% sequence identity as compared to a reference sequence over the window of comparison are also considered to have substantial identity with the reference sequence.

The terms “therapy,” and “treatment,” as used interchangeably herein, refer to an intervention performed with the intention of improving a recipient's status. The improvement can be subjective or objective and is related to the amelioration of the symptoms associated with, preventing the development of, or altering the pathology of a disease, disorder or condition being treated. Thus, the terms therapy and treatment are used in the broadest sense, and include the prevention (prophylaxis), moderation, reduction, and curing of a disease, disorder or condition at various stages. Prevention of deterioration of a recipient's status is also encompassed by the term. Those in need of therapy/treatment include those already having the disease, disorder or condition as well as those prone to, or at risk of developing, the disease, disorder or condition and those in whom the disease, disorder or condition is to be prevented.

The term “ameliorate” or “amelioration” includes the arrest, prevention, decrease, or improvement in one or more the symptoms, signs, and features of the disease being treated, both temporary and long-term.

The term “subject” or “patient” as used herein refers to a mammal in need of treatment.

Administration of the compounds of the invention “in combination with” one or more further therapeutic agents, is intended to include simultaneous (concurrent) administration and consecutive administration. Consecutive administration is intended to encompass administration of the therapeutic agent(s) and the compound(s) of the invention to the subject in various orders.

As used herein, the term “about” refers to a +/−10% variation from the nominal value. It is to be understood that such a variation is always included in any given value provided herein, whether or not it is specifically referred to.

Antisense Molecules

Selection and Characteristics

The antisense oligonucleotides of the present invention are targeted to the gene encoding a mammalian ribonucleotide reductase R1 protein. The sequences of various mammalian ribonucleotide reductase genes are known in the art, for example, the sequence for the human ribonucleotide reductase R1 gene is provided in Bjorklund et al. [P. N. A. S. USA, 90:11322-11326 (1993)]. This and other mammalian R1 sequences are available from the GenBank database maintained by the NCBI.

The antisense oligonucleotides of the present invention comprise at least 7 contiguous nucleotides, or nucleotide analogues, that correspond to a part of the coding region of a mammalian ribonucleotide reductase R1 gene.

Examples of suitable antisense oligonucleotides for use alone or in the combinations of the present invention include those disclosed in U.S. Pat. Nos. 5,998,383 and 6,121,000 (herein incorporated by reference) which are targeted to the ribonucleotide reductase R1 gene. In one embodiment of the present invention, the antisense oligonucleotide comprises at least 7 consecutive nucleotides of the antisense oligonucleotide represented by the sequence:

[SEQ ID NO:1]

5′-CTC TAG CGT CTT AAA GCC GA-3′

The antisense oligonucleotides in accordance with the present invention are selected such that the sequence exhibits the least likelihood of forming duplexes, hair-pins, dimers, or of containing homooligomer/sequence repeats. The oligonucleotide may further contain a GC clamp. One skilled in the art will appreciate that these properties can be determined qualitatively using various computer modelling programs, for example, the program OLIGO® Primer Analysis Software, Version 5.0 (distributed by National Biosciences, Inc., Plymouth, Minn.).

In order to be effective, antisense oligonucleotides are typically between 7 and 100 nucleotides in length. In one embodiment of the present invention, the antisense oligonucleotides are between about 7 to about 50 nucleotides in length. In other embodiments, the antisense oligonucleotides are between about 7 to about 35 nucleotides in length, between about 15 to about 25 nucleotides in length, and about 20 nucleotides in length.

It is understood in the art that an antisense oligonucleotide need not have 100% identity with the complement of its target sequence. The antisense oligonucleotides in accordance with the present invention have a sequence that is at least about 75% identical to the complement of target sequence. In one embodiment of the present invention, the antisense oligonucleotides have a sequence that is at least about 90% identical to the complement of the target sequence. In a related embodiment, they have a sequence that is at least about 95% identical to the complement of target sequence, allowing for gaps or mismatches of several bases. Identity can be determined, for example, by using the BLASTN program of the University of Wisconsin Computer Group (GCG) software or provided on the NCBI website.

The term “antisense oligonucleotides” as used herein includes other oligomeric antisense compounds, including oligonucleotide mimetics, modified oligonucleotides, and chimeric antisense compounds. Chimeric antisense compounds are antisense compounds that contain two or more chemically distinct regions, each made up of at least one monomer unit.

Thus, in the context of this invention, the term “oligonucleotide” refers to an oligomer or polymer of ribonucleic acid (RNA), deoxyribonucleic acid (DNA), or RNA or DNA mimetics. This term, therefore, includes oligonucleotides composed of naturally-occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of antisense compounds useful in this invention include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary antisense oligonucleotides having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In one embodiment of the present invention, the antisense oligonucleotide comprises one or more phosphorothioate internucleotide linkage. In another embodiment, the antisense oligonucleotide comprises phosphorothioate internucleotide linkages that link the four, five or six 3′-terminal nucleotides of the oligonucleotide. In a further embodiment, the antisense oligonucleotide comprises phosphorothioate internucleotide linkages that link all the nucleotides of the oligonucleotide.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulfide, sulfoxide and sulphone backbones; formacetyl and thioformacetyl backbones; methylene formacetyl and thioformacetyl backbones; alkene containing backbones; sulphamate backbones; methyleneimino and methylenehydrazino backbones; sulphonate and sulfonamide backbones; amide backbones; and others having mixed N, O, S and CH₂ component parts.

The present invention also contemplates oligonucleotide mimetics in which both the sugar and the internucleoside linkage of the nucleotide units are replaced with novel groups. The base units are maintained for hybridisation with an appropriate nucleic acid target compound. An example of such an oligonucleotide mimetic, which has been shown to have excellent hybridisation properties, is a peptide nucleic acid (PNA) [Nielsen et al., Science, 254:1497-1500 (1991)]. In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

The present invention also contemplates oligonucleotides comprising “locked nucleic acids” (LNAs), which are novel conformationally restricted oligonucleotide analogues containing a methylene bridge that connects the 2′-O of ribose with the 4′-C (see, Singh et al., Chem. Commun, 1998, 4:455-456). LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al., Tetrahedron, 1998, 54:3607-3630). Studies of mismatched sequences show that LNA obey the Watson-Crick base pairing rules with generally improved selectivity compared to the corresponding unmodified reference strands.

Antisense oligonucleotides containing LNAs have been described (Wahlestedt et al., Proc. Natl. Acad. Sci. U.S.A., 2000, 97:5633-5638), which were efficacious and non-toxic. In addition, the LNA/DNA copolymers were not degraded readily in blood serum and cell extracts.

LNAs form duplexes with complementary DNA or RNA or with complementary LNA, with high thermal affinities. The universality of LNA-mediated hybridization has been emphasized by the formation of exceedingly stable LNA:LNA duplexes (Koshkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

Modified oligonucleotides may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂CH₂OCH₃, also known as 2′-O-(2-methoxyethyl) or 2′-MOE) [Martin et al., Helv. Chim. Acta, 78:486-504 (1995)], 2′-dimethylaminooxyethoxy(O(CH₂)₂ON(CH₃)₂ group, also known as 2′-DMAOE), 2′-methoxy(2′-O—CH₃), 2′-aminopropoxy(2′-OCH₂CH₂CH₂NH₂) and 2′-fluoro (2′-F). In one embodiment of the present invention, the antisense oligonucleotide comprises at least one nucleotide comprising a substituted sugar moiety. In another embodiment, the antisense oligonucleotide comprises at least one 2′-O-(2-methoxyethyl) or 2′-MOE modified nucleotide.

Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include modifications or substitutions to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi, Y. S., (1993) Antisense Research and Applications, pp 276-278, Crooke, S. T. and Lebleu, B., ed., CRC Press, Boca Raton].

Another oligonucleotide modification included in the present invention is the chemical linkage to the oligonucleotide of one or more moieties or conjugates which enhance the activity, cellular distribution or cellular uptake of the oligonucleotide. Such moieties include, but are not limited to, lipid moieties such as a cholesterol moiety [Letsinger et al., Proc. Natl. Acad. Sci. USA, 86:6553-6556 (1989)], cholic acid [Manoharan et al., Bioorg. Med. Chem. Let., 4:1053-1060 (1994)], a thioether, e.g. hexyl-5-tritylthiol [Manoharan et al., Ann. N.Y. Acad. Sci., 660:306-309 (1992); Manoharan et al., Bioorg. Med. Chem. Lett., 3:2765-2770 (1993)], a thiocholesterol [Oberhauser et al., Nucl. Acids Res., 20:533-538 (1992)], an aliphatic chain, e.g. dodecandiol or undecyl residues [Saison-Behmoaras et al., EMBO J., 10:1111-1118 (1991); Kabanov et al., FEBS Lett., 259:327-330 (1990); Svinarchuk et al., Biochimie, 75:49-54 (1993)], a phospholipid, e.g. di-hexadecyl-rac-glycerol or triethylammonium 1,2-di-O-hexadecyl-rac-glycero-3-H-phosphonate [Manoharan et al., Tetrahedron Lett., 36:3651-3654 (1995); Shea et al., Nucl. Acids Res., 18:3777-3783 (1990)], a polyamine or a polyethylene glycol chain [Manoharan et al., Nucleosides & Nucleotides, 14:969-973 (1995)], or adamantane acetic acid [Manoharan et al., Tetrahedron Lett., 36:3651-3654 (1995)], a palmityl moiety [Mishra et al., Biochim. Biophys. Acta, 1264:229-237 (1995)], or an octadecylamine or hexylamino-carbonyl-oxycholesterol moiety [Crooke et al., J. Pharmacol. Exp. Ther., 277:923-937 (1996)].

One skilled in the art will recognise that it is not necessary for all positions in a given oligonucleotide to be uniformly modified. The present invention, therefore, contemplates the incorporation of more than one of the aforementioned modifications into a single oligonucleotide or even at a single nucleoside within the oligonucleotide. The present invention further includes antisense compounds that are chimeric compounds. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridising to the same target region. Cleavage of the RNA target can be routinely detected by gel electrophoresis and, if necessary, associated nucleic acid hybridisation techniques known in the art.

In the context of the present invention, an antisense oligonucleotide is “nuclease resistant” when it has either been modified such that it is not susceptible to degradation by DNA and RNA nucleases or alternatively has been placed in a delivery vehicle which in itself protects the oligonucleotide from DNA or RNA nucleases. Nuclease resistant oligonucleotides include, for example, methyl phosphonates, phosphorothioates, phosphorodithioates, phosphotriesters, and morpholino oligomers. Suitable delivery vehicles for conferring nuclease resistance include, for example, liposomes. In one embodiment of the present invention, the antisense oligonucleotides are nuclease resistant.

The present invention further contemplates antisense oligonucleotides that contain groups for improving the pharmacokinetic properties of the oligonucleotide, or groups for improving the pharmacodynamic properties of the oligonucleotide.

Short Interfering RNA (siRNA) Molecules

The present invention further contemplates that the antisense oligonucleotides may be in the form of siRNA molecules. RNA interference mediated by double-stranded siRNA molecules, which are generated in nature when long double-stranded RNA molecules are cleaved by the action of an endogenous ribonuclease, is known in the art to play an important role in post-transcriptional gene silencing [Zamore, Nature Struc. Biol., 8:746-750 (2001)]. Transfection of mammalian cells with synthetic siRNA molecules having a sequence identical to a target gene has been demonstrated to result in a reduction in the mRNA levels of the target gene [see, for example, Elbashir, et al., Nature, 411:494-498 (2001)]. siRNA molecules are typically 21-22 base pairs in length.

The specificity of siRNA molecules is determined by the binding of the antisense strand of the molecule to its target mRNA. Thus, the antisense oligonucleotides of the present invention can be provided as siRNA molecules which are targeted to a TS gene. As is known in the art, effective siRNA molecules should be less than 30 to 35 base pairs in length to prevent them triggering non-specific RNA interference pathways in the cell via the interferon response. Thus, in one embodiment of the present invention, the siRNA molecules are between about 15 and about 25 base pairs in length. In a related embodiment, they are between 19 and 22 base pairs in length.

The double-stranded siRNA molecules can further comprise poly-T or poly-U overhangs at each end to minimise RNase-mediated degradation of the molecules. In another embodiment of the present invention, the siRNA molecules comprise overhangs at the 3′ and 5′ ends which consist of two thymidine or two uridine residues. Design and construction of siRNA molecules is known in the art [see, for example, Elbashir, et al., Nature, 411:494-498 (2001); Bitko and Barik, BMC Microbiol., 1:34 (2001)]. In addition, kits that provide a rapid and efficient means of constructing siRNA molecules by in vitro transcription are also commercially available (Ambion, Austin, Tex.; New England Biolabs, Beverly, Mass.).

Single-stranded siRNA and short-hairpin siRNA (shRNA) molecules are also known in the art. The present invention contemplates that the antisense oligonucleotides against ribonucleotide reductase R1 can be provided as single-stranded siRNA molecules and as shRNA molecules.

Preparation of the Antisense Oligonucleotides

The antisense oligonucleotides of the present invention can be prepared by conventional techniques well-known to those skilled in the art. For example, the oligonucleotides can be prepared using solid-phase synthesis using commercially available equipment, such as the equipment available from Applied Biosystems Canada Inc., Mississauga, Canada. As is well-known in the art, modified oligonucleotides, such as phosphorothioates and alkylated derivatives, can also be readily prepared by similar methods.

Alternatively, the antisense oligonucleotides of the present invention can be prepared by enzymatic digestion of the naturally occurring ribonucleotide reductase R1 gene by methods known in the art.

Antisense oligonucleotides can also be prepared through the use of recombinant methods in which expression vectors comprising nucleic acid sequences that encode the antisense oligonucleotides are expressed in a suitable host cell. Such expression vectors can be readily constructed using procedures known in the art. Examples of suitable vectors include, but are not limited to, plasmids, phagemids, cosmids, bacteriophages, baculoviruses and retroviruses, and DNA viruses. One skilled in the art will understand that selection of the appropriate host cell for expression of the antisense oligonucleotide will be dependent upon the vector chosen. Examples of host cells include, but are not limited to, bacterial, yeast, insect, plant and mammalian cells.

One skilled in the art will also understand that the expression vector may further include regulatory elements, such as transcriptional elements, required for efficient transcription of the antisense oligonucleotide sequences. Examples of regulatory elements that can be incorporated into the vector include, but are not limited to, promoters, enhancers, terminators, and polyadenylation signals. One skilled in the art will appreciate that selection of suitable regulatory elements is dependent on the host cell chosen for expression of the antisense oligonucleotide and that such regulatory elements may be derived from a variety of sources, including bacterial, fungal, viral, mammalian or insect genes.

In accordance with the present invention, the expression vectors can be introduced into a suitable host cell or tissue by one of a variety of methods known in the art. Such methods can be found generally described in Sambrook et al., 1992; Ausubel et al., 1989; Chang et al., 1995; Vega et al., 1995; and Vectors: A Survey of Molecular Cloning Vectors and Their Uses (1988) and include, for example, stable or transient transfection, lipofection, electroporation, and infection with recombinant viral vectors.

Chemotherapeutic Agents

When the antisense oligonucleotides of the present invention are used in combination with one or more chemotherapeutic agents, the chemotherapeutic agent can be selected from a wide range of cancer chemotherapeutic agents known in the art. Known chemotherapeutic agents include those that are specific for the treatment of a particular type of cancer as well as those that are applicable to a range of cancers, such as doxorubicin, capecitabine, mitoxantrone, irinotecan (CPTI-11) and gemcitabine. Etoposide is generally applicable in the treatment of leukaemias (including acute lymphocytic leukaemia and acute myeloid leukaemia), germ cell tumours, Hodgkin's disease and various sarcomas. Cytarabine (Ara-C) is also applicable in the treatment of various leukaemias, including acute myeloid leukaemia, meningeal leukaemia, acute lymphocytic leukaemia, chronic myeloid leukaemia, erythroleukaemia, as well as non-Hodgkin's lymphoma.

The present invention contemplates the use of both types of chemotherapeutic agent in conjunction with the antisense oligonucleotides. Exemplary chemotherapeutics that can be used alone or in various combinations for the treatment specific cancers are provided in Table 1. One skilled in the art will appreciate that many other chemotherapeutics are available and that the following list is representative only.

TABLE 1
Exemplary Chemotherapeutics used in the Treatment of Some
Common Cancers

| CANCER | CHEMOTHERAPEUTIC | |
|---|---|---|
| Acute lymphocytic | Pegaspargase (e.g. Oncaspar ®) | L-asparaginase |
| leukaemia (ALL) | Interleukin-2 (e.g. Proleukin ®) | Cytarabine |
| Acute myeloid | Cytarabine | Idarubicin |
| leukaemia (AML) | | |
| Brain cancer | Procarbazine (e.g. Matulane ®) | |
| Breast cancer | Capecitabine (e.g. Xeloda ®) | Cyclophosphamide |
| | 5-fluorouracil (5-FU) | Carboplatin |
| | Paclitaxel (e.g. Taxol ®) | Cisplatin |
| | Docetaxel (e.g. Taxotere ®) | Ifosfamide |
| | Epi-doxorubicin (epirubicin) | Doxorubicin (e.g. Adriamycin ®) |
| | Trastuzumab (Herceptin ®) | Tamoxifen |
| Chronic myeloid | Low-dose Interferon (IFN)-alpha | |
| leukaemia (CML) | Cytarabine | |
| Colon cancer | Edatrexate (10-ethyl-10-deaza-aminopterin) | |
| | Methyl-chloroethyl-cyclohexyl-nitrosourea | |
| | 5-fluorouracil (5-FU) | Levamisole |
| | Fluorodeoxyuridine (FUdR) | Vincristine |
| | Capecitabine (e.g. Xeloda ®) | Oxaliplatin |
| | Gemcitabine (e.g. Gemzar ®) | |
| Colorectal cancer | Irinotecan (CPT-11, e.g. Camptosar ®) | |
| | Loperamide (e.g. Imodium ®) | |
| | Topotecan (e.g. Hycamtin ®) | |
| | Capecitabine (e.g. Xeloda ®) | Oxaliplatin |
| | 5-fluorouracil (5-FU) | |
| Gall bladder | 5-fluorouracil (5-FU) | |
| Genitourinary cancer | Docetaxel (e.g. Taxotere ®) | |
| Head and neck | Docetaxel (e.g. Taxotere ®) | Cisplatin |
| cancer | | |
| Non-Hodgkin's | Procarbazine (e.g. Matulane ®) | Cytarabine |
| Lymphoma | Rituximab (e.g. Rituxan ®) | Etoposide |
| Non-small-cell lung | Vinorelbine Tartrate (e.g. Navelbine ®) | |
| (NSCL) cancer | Irinotecan (CPT-11, e.g. Camptosar ®) | |
| | Docetaxel (e.g. Taxotere ®) | Paclitaxel (e.g. Taxol ®) |
| | Gemcitabine (e.g. Gemzar ®) | Topotecan |
| Oesophageal cancer | Porfimer Sodium (e.g. Photofrin ®) | |
| | Cisplatin | |
| Ovarian cancer | Irinotecan (CPT-11, e.g. Camptosar ®) | |
| | Topotecan (e.g. Hycamtin ®) | |
| | Docetaxel (e.g. Taxotere ®) | Paclitaxel (e.g. Taxol ®) |
| | Gemcitabine (e.g. Gemzar ®) | Amifostine (e.g. Ethyol ®) |
| Pancreatic cancer | Irinotecan (CPT-11, e.g. Camptosar ®) | |
| | Gemcitabine (e.g. Gemzar ®) | 5-fluorouracil (5-FU) |
| Promyelocytic | Tretinoin (e.g. Vesanoid ®) | |
| leukaemia | | |
| Prostate cancer | Goserelin Acetate (e.g. Zoladex ®) | |
| | Mitoxantrone (e.g. Novantrone ®) | |
| | Prednisone (e.g. Deltasone ®) | Liarozole |
| | Nilutamide (e.g. Nilandron ®) | Flutamide (e.g. Eulexin ®) |
| | Finasteride (e.g. Proscar ®) | Terazosin (e.g. Hytrin ®) |
| | Doxazosin (e.g. Cardura ®) | Cyclophosphamide |
| | Docetaxel (e.g. Taxotere ®) | Estramustine |
| Renal cancer | Capecitabine (e.g. Xeloda ®) | |
| | Gemcitabine (e.g. Gemzar ®) | |
| | Interleukin-2 (e.g. Proleukin ®) | |
| Small cell lung | Cyclophosphamide | Vincristine |
| cancer | Doxorubicin | Etoposide |
| Solid tumours | Gemicitabine (e.g. Gemzar ®) | Cyclophosphamide |
| | Capecitabine (e.g. Xeloda ®) | Ifosfamide |
| | Paclitaxel (e.g. Taxol ®) | Cisplatin |
| | Docetaxel (e.g. Taxotere ®) | Carboplatin |
| | Epi-doxorubicin (epirubicin) | Doxorubicin (e.g. Adriamycin ®) |
| | 5-fluorouracil (5-FU) | |

As indicated above, combinations of chemotherapeutics may be employed. Combination therapies using standard cancer chemotherapeutics are well known in the art and such combinations also can be used in conjunction with the antisense oligonucleotides of the invention.

Exemplary combination therapies include for the treatment of breast cancers the combination of epirubicin with paclitaxel or docetaxel, or the combination of doxorubicin or epirubicin with cyclophosphamide. Polychemotherapeutic regimens are also useful and may consist, for example, of doxorubicin/cyclophosphamide/5-fluorouracil or cyclophosphamide/epirubicin/5-fluorouracil. Many of the above combinations are useful in the treatment of a variety of other solid tumours.

Combinations of etoposide with either cisplatin or carboplatin are used in the treatment of small cell lung cancer. In the treatment of stomach or oesophageal cancer, combinations of doxorubicin or epirubicin with cisplatin and 5-fluorouracil are useful. For colorectal cancer, CPT-11 in combination with 5-fluorouracil-based drugs, or oxaliplatin in combination with 5-fluorouracil-based drugs can be used. Oxaliplatin may also be used in combination with capecitabine.

Other examples include the combination of cyclophosphamide, doxorubicin, vincristine and prednisone in the treatment of non-Hodgkin's lymphoma; the combination of doxorubicin, bleomycin, vinblastine and dacarbazine (DTIC) in the treatment of Hodgkin's disease and the combination of cisplatin or carboplatin with any one, or a combination, of gemcitabine, paclitaxel, docetaxel, vinorelbine or etoposide in the treatment of non-small cell lung cancer.

Various sarcomas are treated by combination therapy, for example, for osteosarcoma combinations of doxorubicin and cisplatin or methotrexate with leucovorin are used; for advanced sarcomas etoposide can be used in combination with ifosfamide; for soft tissue sarcoma doxorubicin or dacarbazine can be used alone or, for advanced sarcomas doxorubicin can be used in combination with ifosfamide or dacarbazine, or etoposide in combination with ifosfamide.

Ewing's sarcoma/peripheral neuroectodermal tumour (PNET) or rhabdomyosarcoma can be treated using etoposide and ifosfamide, or a combination of vincristine, doxorubicin and cyclophosphamide.

The alkylating agents cyclophosphamide, cisplatin and melphalan are also often used in combination therapies with other chemotherapeutics in the treatment of various cancers.

Examples of suitable combinations of the antisense oligonucleotide and one or more chemotherapeutic agent include, but are not limited to, a combination of the antisense oligonucleotide
with capecitabine, alone or in combination with other chemotherapeutics, for the treatment of solid tumours including, but not limited to, breast cancer, renal cancer, colon cancer, colorectal cancer and pancreatic cancer, for example, a combination of capecitabine and oxaliplatin for the treatment of colorectal cancer, colon cancer and pancreatic cancer or a combination of capecitabine and gemcitabine for the treatment of colon cancer;
with a combination of carboplatin and paclitaxel for the treatment of metastatic cancers;
with cisplatin, alone or in combination with other chemotherapeutics, for the treatment of head and neck cancer, oesophageal cancer, lung cancer, ovarian cancer and cervical cancer, for example a combination of cisplatin and irinotecan for the treatment of small-cell lung carcinoma (SCLC);
with cytarabine, alone or in combination with other chemotherapeutics, for the treatment of acute myeloid leukaemia (AML) and chronic myeloid leukaemia (CML), for example, a combination of cytarabine, fludarabine and filgrastim for the treatment of CML, or a combination of cytarabine, mitoxantrone and etoposide for the treatment of AML;
with dacarbazine for the treatment of melanoma;
with docetaxel, alone or in combination with other chemotherapeutics, for the treatment of solid tumours, including, but not limited to, non-small cell lung carcinoma (NSCLC), breast cancer, prostate cancer and cancer of the genitourinary tract;
with 5-FU, alone or in combination with other chemotherapeutics, for the treatment of renal cancer, pancreatic cancer, and cancers of the gall bladder or biliary ducts;
with gemcitabine, alone or in combination with other chemotherapeutics, for the treatment of solid tumours, including, but not limited to, NSCLC, breast cancer and renal cancer, for example, a combination of gemcitabine and oxaliplatin for the treatment of breast cancer;
with hydroxyurea, alone or in combination with other chemotherapeutics, for the treatment of cervical cancer;
with idarubicin, alone or in combination with other chemotherapeutics, for the treatment of AML;
with irinotecan, alone or in combination with other chemotherapeutics, for the treatment of pancreatic cancer and colon cancer;
with mitoxantrone, alone or in combination with other chemotherapeutics, for the treatment of prostate cancer and colon cancer, for example, a combination of mitoxantrone and prednisone for the treatment of prostate cancer;
with taxol, alone or in combination with other chemotherapeutics, for the treatment of ovarian cancer and breast cancer, and
with vinblastine, alone or in combination with other chemotherapeutics, for the treatment of renal cancer.
   Efficacy of the Antisense Oligonucleotides and Combinations

The antisense oligonucleotides of the present invention can be initially tested, alone or in combination with other chemotherapeutic(s), for their ability to attenuate the growth and/or metastasis of cancer cells in vitro and/or in vivo. Methods of testing potential anti-cancer compounds are known in the art. Exemplary, non-limiting tests are provided below and in the Examples included herein.

1. In Vitro Testing

Initial determinations of the efficacy of the antisense oligonucleotides alone, or in combination with one or more chemotherapeutic agents (“combinations”), may be made using in vitro techniques if required.

For example, the antisense oligonucleotides or combinations of the antisense oligonucleotides with one or more chemotherapeutic agents can be tested in vitro by determining their ability to inhibit anchorage-independent growth of tumour cells. Anchorage-independent growth is known in the art to be a good indicator of tumourigenicity. In general, anchorage-independent growth is assessed by plating cells from an appropriate cancer cell-line onto soft agar and determining the number of colonies formed after an appropriate incubation period. Growth of cells treated with the antisense oligonucleotides alone or combinations can then be compared with that of cells treated with an appropriate control (such as cells treated with a scrambled control oligonucleotide or a known chemotherapeutic, or untreated cells) and with that of untreated cells.

Typically in vitro testing of the antisense oligonucleotides and combinations is conducted in a human cancer cell-line. Examples of suitable cancer cell-lines for in vitro testing of the antisense oligonucleotides or combinations of the present invention are known in the art and include those described in the Examples provided herein.

If necessary, the toxicity of the antisense oligonucleotides and combinations can also be initially assessed in vitro using standard techniques. For example, human primary fibroblasts can be treated in vitro with the oligonucleotide in the presence of a commercial lipid carrier such as lipofectamine. Cells are then tested at different time points following treatment for their viability using a standard viability assay, such as the trypan-blue exclusion assay. Cells are also assayed for their ability to synthesize DNA, for example, using a thymidine incorporation assay, and for changes in cell cycle dynamics, for example, using a standard cell sorting assay in conjunction with a fluorocytometer cell sorter (FACS).

2. In Vivo Testing

The ability of the antisense oligonucleotides and combinations to inhibit tumour growth or proliferation in vivo can be determined in an appropriate animal model using standard techniques known in the art (see, for example, Enna, et al., Current Protocols in Pharmacology, J. Wiley & Sons, Inc., New York, N.Y.).

In general, current animal models for screening anti-tumour compounds are xenograft models, in which a human tumour has been implanted into an animal. Examples of xenograft models of human cancer include, but are not limited to, human solid tumour xenografts in mice, implanted by sub-cutaneous injection and used in tumour growth assays; human solid tumour isografts in mice, implanted by fat pad injection and used in tumour growth assays; experimental models of lymphoma and leukaemia in mice, used in survival assays, and experimental models of lung metastasis in mice. Representative, non-limiting examples are provided in Table 2 and in the Examples provided herein.

For example, the antisense oligonucleotides and combinations can be tested in vivo on solid tumours using mice that are subcutaneously grafted bilaterally with a pre-determined amount of a tumour fragment on day 0. The animals bearing tumours are mixed before being subjected to the various treatments and controls. In the case of treatment of advanced tumours, tumours are allowed to develop to the desired size, animals having insufficiently developed tumours being eliminated. The selected animals are distributed at random into groups that will undergo the treatments or act as controls. Suitable groupings would be, for example, those receiving the combination of the invention, those receiving the antisense alone, those receiving the chemotherapeutic agent(s) alone and those receiving no treatment. Animals not bearing tumours may also be subjected to the same treatments as the tumour-bearing animals in order to be able to dissociate the toxic effect from the specific effect on the tumour. Chemotherapy generally begins from 3 to 22 days after grafting, depending on the type of tumour, and the animals are observed every day. The antisense oligonucleotides or combinations of the present invention can be administered to the animals, for example, by bolus infusion. The different animal groups are weighed about 3 or 4 times a week until the maximum weight loss is attained, after which the groups are weighed at least once a week until the end of the trial.

The tumours are measured about 2 or 3 times a week until the tumour reaches a pre-determined size and/or weight, or until the animal dies if this occurs before the tumour reaches the pre-determined size/weight. The animals are then sacrificed and the tissue histology, size and/or proliferation of the tumour assessed.

For the study of the effect of the antisense oligonucleotides and combinations on leukaemias, the animals are grafted with a particular number of cells, and the anti-tumour activity is determined by the increase in the survival time of the treated mice relative to the controls.

To study the effect of the antisense oligonucleotides and combinations of the present invention on tumour metastasis, tumour cells are typically treated with the composition ex vivo and then injected into a suitable test animal. The spread of the tumour cells from the site of injection is then monitored over a suitable period of time by standard techniques.

In vivo toxic effects of the oligonucleotides can be evaluated by measuring their effect on animal body weight during treatment and by performing haematological profiles and liver enzyme analysis after the animal has been sacrificed.

TABLE 2
Examples of xenograft models of human cancer

| Cancer Model | Cell Type |
|---|---|
| Tumour Growth Assay | Prostate (PC-3, DU145) |
| Human solid tumour xenografts | Breast (MDA-MB-231, MVB-9) |
| in mice (sub-cutaneous | Colon (HT-29) |
| injection) | Lung (NCI-H460, NCI-H209) |
| | Pancreatic (ASPC-1, SU86.86) |
| | Pancreatic: drug resistant (BxPC-3) |
| | Skin (A2058, C8161) |
| | Cervical (SIHA, HeLa-S3) |
| | Cervical: drug resistant (HeLa S3-HU- |
| | resistance) |
| | Liver (HepG2) |
| | Brain (U87-MG) |
| | Renal (Caki-1, A498) |
| | Ovary (SK-OV-3) |
| Tumour Growth Assay | Breast: drug resistant (MDA-CDDP-S4, |
| Human solid tumour isografts in | MDA-MB435-To. 1) |
| mice (fat pad injection) | |
| Survival Assay | Human: Burkitts lymphoma |
| Experimental model of | (Non-Hodgkin's) (raji) |
| lymphoma and leukaemia in | Murine: erythroleukaemia (CB7 Friend |
| mice | retrovirus-induced) |
| Experimental model of lung | Human: melanoma (C8161) |
| metastasis in mice | Murine: fibrosarcoma (R3) |

Pharmaceutical Compositions

For the treatment of cancer in a mammal, the antisense oligonucleotide may be administered as a pharmaceutical composition comprising the antisense oligonucleotide in admixture with an appropriate pharmaceutically physiologically acceptable carrier, diluent, excipient or vehicle. The pharmaceutical compositions may also be formulated to contain the antisense oligonucleotide and one or more other chemotherapeutic agents for concurrent administration to a patient, where appropriate.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous, intravenous, intramuscular, intrasternal, intrathecal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryi distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol, water, Ringer's solution, lactated Ringer's solution or isotonic sodium chloride solution. Other examples of acceptable vehicles and solvents that may be employed include, but are not limited to, sterile, fixed oils which are conventionally employed as a solvent or suspending medium, and a variety of bland fixed oils including, for example, synthetic mono- or diglycerides. In addition, fatty acids such as oleic acid find use in the preparation of injectables. Injectable compositions are also suitable for administration by continuous infusion.

In one embodiment of the present invention, the antisense oligonucleotide is formulated as an injectable composition.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philidelphia, Pa. (2000) (formerly “Remingtons Pharmaceutical Sciences”).

Use of the Antisense Oligonucleotides and Combinations

The antisense oligonucleotides of the present invention and combinations comprising an antisense oligonucleotide and one or more chemotherapeutic agents can be used in the treatment of a variety of cancers. In one embodiment of the present invention, the combination is more effective in reducing the growth and/or metastasis of cancer cells than the chemotherapeutic agent(s) alone. The antisense oligonucleotides and combinations can also be used to effectively treat drug resistant tumours.

Examples of cancers which may be may be treated, stabilised, or prevented in accordance with the present invention include, but are not limited to leukaemia, carcinomas, adenocarcinomas, sarcomas, lymphomas and melanomas. Carcinomas, adenocarcinomas and sarcomas are also frequently referred to as “solid tumours,” examples of commonly occurring solid tumours include, but are not limited to, cancer of the brain, breast, cervix, colon, head and neck, kidney, lung, ovary, pancreas, prostate, lung, stomach and uterus, and colorectal cancer. Lymphomas are also considered to be solid tumours.

The term “leukaemia” refers broadly to progressive, malignant diseases of the blood-forming organs. Leukaemia is typically characterised by a distorted proliferation and development of leukocytes and their precursors in the blood and bone marrow but can also refer to malignant diseases of other blood cells such as erythroleukaemia, which affects immature red blood cells. Leukaemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukaemia includes, for example, acute nonlymphocytic leukaemia, chronic lymphocytic leukaemia, acute granulocytic leukaemia, chronic granulocytic leukaemia, acute promyelocytic leukaemia, acute myeloid leukaemia (AML), chronic myeloid leukaemia (CML), adult T-ell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthile cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

Commonly occurring carcinomas that may be treated with the antisense oligonucleotides of the present invention, include, for example, pancreatic, ovarian, lung, liver, renal and cervical carcinomas.

The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas, colon and prostate.

The term “sarcoma” generally refers to a tumour which originates in connective tissue, such as muscle, bone, cartilage or fat, and is made up of a substance like embryonic connective tissue and is generally composed of closely packed cells embedded in a fibrillar or homogeneous substance. Sarcomas include soft tissue sarcomas, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumour sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumour arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The antisense oligonucleotides of the present invention can also be used in the treatment of lymphomas including Hodgkin's and non-Hodgkin's lymphomas and brain cancers including primary brain tumours, gliomas, glioblastoma multiforme; malignant astrocytomas; oligdendroglioma; ependymoma; low-grade astrocytomas; meningioma; mesenchymal tumours; pituitary tumours; nerve sheath tumours such as schwannomas; central nervous system lymphoma; medulloblastoma; primitive neuroectodermal tumours; neuron and neuron/glial tumours; craniopharyngioma; germ cell tumours and choroid plexus tumours. Additional cancers include multiple myeloma, neuroblastoma, rhabdomyosarcoma, primary thrombocytosis, primary macroglobulinemia, small-cell lung tumours, malignant pancreatic insulanoma, malignant carcinoid, urinary bladder cancer, premalignant skin lesions, testicular cancer, thyroid cancer, oesophageal cancer, genitourinary tract cancer, malignant hypercalcemia, endometrial cancer, adrenal cortical cancer and mesothelioma.

The cancer may be indolent or it may be aggressive. The antisense oligonucleotides are useful in the treatment of refractory cancers, advanced cancers, recurrent cancers, relapsed and metastatic cancers. One skilled in the art will appreciate that many of these categories may overlap, for example, aggressive cancers are typically also advanced and/or metastatic.

“Aggressive cancer,” as used herein, refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types. A “refractory” cancer or tumour refers to a cancer or tumour that has not responded to treatment. “Advanced cancer,” refers to overt disease in a patient, wherein such overt disease is not amenable to cure by local modalities of treatment, such as surgery or radiotherapy. Advanced disease may refer to a locally advanced cancer or it may refer to metastatic cancer. The term “metastatic cancer” refers to cancer that has spread from one part of the body to another. Advanced cancers may also be unresectable, that is, they have spread to surrounding tissue and cannot be surgically removed.

The antisense oligonucleotides may also be used to treat drug resistant cancers, including multidrug resistant tumours. As is known in the art, the resistance of cancer cells to chemotherapy is one of the central problems in the management of cancer.

Certain cancers, such as prostate and breast cancer, can be treated by hormone therapy, i.e. with hormones or anti-hormone drugs that slow or stop the growth of certain cancers by blocking the body's natural hormones. Such cancers may develop resistance, or be intrinsically resistant, to hormone therapy. The present invention further contemplates the use of the antisense oligonucleotide in the treatment of these “hormone-resistant” or “hormone-refractory” cancers.

In one embodiment of the present invention, the antisense oligonucleotide alone, or in combination with one or more chemotherapeutic, is used in the treatment of solid tumours including metastatic, advanced, drug- or hormone-resistant versions of solid tumours. In another embodiment, the solid tumour is a renal tumour, breast tumour, lung tumour, prostate tumour, colon tumour, melanoma, ovarian tumour, cervical tumour, brain tumour, liver tumour, colorectal tumour, pancreatic tumour, genitourinary tumour, gall bladder tumour, head and neck tumour, oesophageal tumour biliary duct tumour, a lymphoma, or a sarcoma, including a metastatic, advanced, drug- or hormone-resistant version thereof. In a further embodiment, the solid tumour is an ovarian tumour, a renal tumour, a brain tumour, or a sarcoma, including a metastatic, advanced, or drug-resistant version thereof.

In an alternate embodiment, the antisense oligonucleotide alone, or in combination with one or more chemotherapeutic, is used in the treatment of a leukaemia, including a metastatic, advanced or drug-resistant version thereof.

Administration of the Antisense Oligonucleotides

The dose of the antisense oligonucleotide of the present invention to be administered to a patient should be a sufficient amount to effect a beneficial therapeutic response in the patient over time, i.e. an “effective amount.” Such a beneficial therapeutic response may be, for example, stabilisation of the disease, tumour shrinkage, decreased time to progression or prolonged survival. The dose will be determined by the efficacy of the particular oligonucleotide employed, the type of cancer to be treated and the condition of the patient to be treated, as well as the body weight or surface area of the patient. Appropriate doses can be readily determined by a skilled practitioner.

Typically, antisense oligonucleotides are administered systemically to patients. Administration can be accomplished by bolus injection as a single dose or as divided doses, or by continuous infusion over an appropriate period of time.

In one embodiment of the present invention, the antisense oligonucleotides are administered by continuous infusion. In another embodiment, the antisense oligonucleotides are administered by continuous intravenous infusion.

As indicated above, the dosage of the antisense oligonucleotide to be administered will be dependent upon the type of cancer to be treated and the size of the patient and can be readily determined by a skilled practitioner. By way of example only, for the antisense oligonucleotide represented by SEQ ID NO:1, appropriate doses determined by Phase I clinical trials are between about 18.5 mg/m²/day and about 222 mg/m²/day. In one embodiment, the dose of antisense oligonucleotide is between about 37 mg/m²/day and about 222 mg/m²/day. In another embodiment, the dose of antisense oligonucleotide is between about 74 mg/m²/day and about 185 mg/m²/day. In further embodiments, the dose of antisense oligonucleotide is between about 100 mg/m²/day and about 185 mg/m²/day and between about 148 mg/m²/day and about 185 mg/m²/day. In further embodiments, the dose of the antisense oligonucleotide is between about 6.0 mg/m²/day and about 356.5 mg/m²/day. In other embodiments, the dose of antisense oligonucleotide is between about 6.0 mg/m²/day and about 274.2 mg/m²/day, between about 48.0 mg/m²/day and about 274.2 mg/m²/day and between about 96.0 mg/m²/day and about 274.2 mg/m²/day. In another embodiment, the dose of antisense oligonucleotide is between about 96.0 mg/m²/day and about 210.9 mg/m²/day. In further embodiments, the dose of antisense oligonucleotide is between about 96.0 mg/m²/day and about 162.2 mg/m²/day and between about 124.8 mg/m²/day and about 210.9 mg/m²/day. Other exemplary doses for SEQ ID NO:1 include doses between about 0.16 mg/kg/day and about 10 mg/kg/day, between about 2 mg/kg/day and about 10 mg/kg/day, between about 3 mg/kg/day and about 8 mg/kg/day and between about 3 mg/kg/day and about 5 mg/kg/day.

Treatment regimens can be designed such that the antisense oligonucleotide is administered to the patient in cycles. Treatment with antisense oligonucleotide in accordance with the present invention, therefore, may be part of a treatment regimen that involves one cycle of administration or more than one cycle. Typically, a cycle is between about 1 and about 4 weeks. Exemplary dosing schedules comprise one or more cycle of 21 days continuous infusion followed by 7 days of rest or one or more cycles of 14 days continuous infusion followed by 7 days of rest. Further examples are provided in the Examples section herein. Other treatment regimens can be readily determined by the skilled practitioner. Between one and sixteen cycles of treatment are contemplated, however, additional cycles may be incorporated into the treatment regimen as necessary.

The present invention contemplates the use of the antisense oligonucleotides, alone or in combination with one or more other chemotherapeutic agents, to treat patients who have undergone prior chemotherapy. Thus, in one embodiment of the invention, the antisense oligonucleotides are used as a second or subsequent (for example, third or fourth) line of therapy. In an alternate embodiment, the antisense oligonucleotides are used to treat patients who have already undergone more than one course of prior chemotherapy. The antisense oligonucleotides, alone or in combination with one or more other chemotherapeutic agents, may also be used as a first line of therapy in the treatment of patients for whom standard chemotherapy is not suitable.

As indicated above, the antisense oligonucleotide can be administered to the patient in conjunction with one or more chemotherapeutic agents. In such combination therapy, the antisense oligonucleotide can be administered prior to, or after, administration of the one or more other chemotherapeutic agents, or it can be administered concurrently. The one or more chemotherapeutic may be administered systemically, for example, by bolus injection or continuous infusion, or it may be administered orally.

The one or more other chemotherapeutic may also be administered in cycles, which may or may not overlap with the cycles of administration for the antisense oligonucleotide. When the antisense oligonucleotide is administered prior to the one or more other chemotherapeutic agents, the length of time between the initiation of administration of the antisense oligonucleotide and the other agent(s) will depend on the mode of administration, the size of the patient and the nature of the other agent(s) being administered. Similarly, if the antisense oligonucleotide and the one or more other chemotherapeutic agents are administered concurrently, administration of the compounds may be initiated at the same time, or administration of the other chemotherapeutic(s) may be initiated at a suitable time prior to or after administration of the antisense oligonucleotide is initiated. Appropriate treatment regimens can be readily determined by the skilled practitioner.

Appropriate doses and treatment regimens for standard chemotherapeutics for the treatment of a variety of cancers are well known in the art. The following are provided by way of example only and are not intended to limit the scope of the present invention in any way.

Capecitabine can be administered at a dose of between about 500 and about 2000 mg/m²/day. Capecitabine is typically administered orally. Administration of the daily amount may be via a single dose or divided doses. Exemplary doses would be between about 500-1500 mg/m²/day, between about 600-1000 mg/m²/day, and between about 1100-2000 mg/m²/day depending on the type of cancer being treated. In one embodiment, capecitabine at a dose of between 850 and 1700 mg/m²/day is used in conjunction with the antisense oligonucleotide. In another embodiment, doses of 850, 1250 and 1660 mg/m²/day are used.

Cytarabine can be administered at various doses between about 5 and about 3000 mg/m²/day depending on the type of cancer being treated and the dosing schedule employed. Administration of the daily amount of cytarabine may be via a single dose, divided dose or continuous infusion. Exemplary doses would be between about 500-1000 mg/m²/day, between about 1000-2000 mg/m²/day and between about 4000-6000 mg/m²/day. In one embodiment, cytarabine at a dose of between about between about 4000-6000 mg/m²/day is used in conjunction with the antisense oligonucleotide.

For some indications, cytarabine can be administered intrathecally at a dose of between about 5-75 mg/m²/day and between about 100-200 mg/m²/day, depending on the type of cancer being treated and the dosing schedule employed. Thus, for certain cancers, cytarabine is used at a dose of between about 5-75 mg/m²/day in conjunction with the antisense oligonucleotide.

Docetaxel can be administered at a dose of between about 20 and about 100 mg/m² per one dose. Exemplary doses would be between about 30-35 mg/m², between about 30-36 mg/m², between about 60-75 mg/m², between about 40-80 mg/m² and between about 60-100 mg/m² depending on the type of cancer being treated and the dosing schedule employed. In one embodiment, docetaxel at a dose of between about 60 mg/m² and about 75 mg/m² is used in conjunction with the antisense oligonucleotide. In another embodiment, the docetaxel at a dose of between aobut 45 mg/m² to about 75 mg/m² is used in conjunction with the antisense oligonucleotide.

Paclitaxel can be administered at a dose of between about 50 mg/m² and about 200 mg/m². Paclitaxel may be administered via intermittent infusion at a dose of between about 90 mg/m² to about 175 mg/m², or continuous infusion, at a dose of between about 50 mg/m² to about 135 mg/m² depending on the cancer treated and the dosing scheduled employed. In one embodiment, paclitaxel at a dose of between about 50 mg/m² and about 200 mg/m² is used in conjunction with the antisense oligonucleotide. In another embodiment, paclitaxel at a dose of between about 50 mg/m² and about 135 mg/m² is used in conjunction with the antisense oligonucleotide. In a further embodiment, paclitaxel at a dose of between about 90 mg/m² and about 175 mg/m² is used in conjunction with the antisense oligonucleotide.

Irinotecan (CPT-11) can be administered at a dose of between about 75 mg/m² to about 700 mg/m² depending on the dosing schedule employed. Irinotecan is typically administered intravenously using single or divided doses. Exemplary single doses would be between about 250 mg/m² to about 350 mg/m² and between about 75 mg/m² to about 125 mg/m². In one embodiment, irinotecan at a dose of between about 75 mg/m² and about 700 mg/m² is used in conjunction with the antisense oligonucleotide. In other embodiments, irinotecan at a dose of between about 75 mg/m² and about 125 mg/m², and between about 250 mg/m² and about 350 mg/m² is used in conjunction with the antisense oligonucleotide.

Cisplatin can be administered at a dose of between about 20 mg/m² to about 100 mg/m² depending on the dosing schedule employed. Exemplary doses of cisplatin would be between about 20 mg/m²/day to about 60 mg/m²/day. Lower daily doses of about 20 to mg/m²/day to about 35 mg/m²/day may be administered with less intensive hydration. Depending on the dosing schedule employed, cisplatin can be administered at a dose of between about 75 mg/m² to about 100 mg/m². In one embodiment, cisplatin at a dose of between about 25 mg/m²/day to about 60 mg/m²/day is used in conjunction with the antisense oligonucleotide. In another embodiment, cisplatin at a dose of between about 20 mg/m² to about 100 mg/m² is used in conjunction with the antisense oligonucleotide. In further embodiments, doses of cisplatin are between about 20 mg/m²/day to about 60 mg/m²/day and between about 75 mg/m² and about 100 mg/m².

Single doses of mitomycin C are typically between about 10 mg/m² to about 20 mg/m². Mitomycin C is typically administered via intravenous infusion. In some indications mitomycin C can be administered at lower daily doses of about 2 mg/m²/day depending on the dosing schedule employed. In one embodiment, mitomycin C is used at a dose of between about 10 mg/m² to about 20 mg/m², in conjunction with the antisense oligonucleotide. In other embodiments, mitomycin C is used at a daily dose of about 2 mg/m²/day.

Single dose units of gemcitabine are typically between about 100 and about 2500 mg/m². Exemplary dose units suitable for use with the antisense oligonucleotides would be between about 400-1000 mg/m², between about 600-1000 mg/r², between about 800-1000 mg/m², between about 500-1250 mg/m², between about 750-1200 mg/m², between about 800-1250 mg/m², between about 1000-1200 mg/m², between about 1250-2500 mg/m², depending on the type of cancer being treated and the dosing schedule employed. The dose maybe administered, for example, weekly or biweekly. In one embodiment, a weekly unit dose of between about 400-1000 mg/m² gemcitabine is used in conjunction with the antisense oligonucleotide.

For some indications, gemcitabine can also be administered at lower doses, for example, between about 100 to about 400 mg/m²/day depending on the type of cancer being treated.

Oxaliplatin can be administered at a dose of between about 30 and about 135 mg/m²/day. Administration of the daily amount of oxaliplatin may be via a single dose or divided doses, or by continuous infusion. Exemplary doses would be between about 80-100 mg/m²/day and between about 85-135 mg/m²/day depending on the type of cancer being treated and the dosing schedule employed. In one embodiment, oxaliplatin at a dose of about 130 mg/m²/day is used in conjunction with the antisense oligonucleotide.

It is to be understood, however, that the above exemplary dosages and frequencies of administration may be adapted to the circumstances in accordance with known practices in the art for the treatment of different cancers.

Clinical Trials in Cancer Patients

One skilled in the art will appreciate that, following the demonstrated effectiveness of the antisense oligonucleotides alone or combinations of the present invention in vitro and in animal models, they should be tested in Clinical Trials in order to further evaluate their efficacy in the treatment of cancer and to obtain regulatory approval for therapeutic use. As is known in the art, clinical trials progress through phases of testing, which are identified as Phases I, II, III, and IV.

Initially the antisense oligonucleotides alone or combinations will be evaluated in a Phase I trial. Typically Phase I trials are used to determine the best mode of administration (for example, by pill or by injection), the frequency of administration, and the toxicity for the compounds. Phase I studies frequently include laboratory tests, such as blood tests and biopsies, to evaluate the effects of a compound in the body of the patient. For a Phase I trial, a small group of cancer patients are treated with a specific dose of the antisense oligonucleotide and the one or more chemotherapeutic agent(s). During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to evaluate further the effectiveness and safety of the antisense oligonucleotides alone or combinations. In Phase II trials, the antisense oligonucleotides alone or the combination is administered to groups of patients with either one specific type of cancer or with related cancers, using the dosage found to be effective in Phase I trials.

Phase III trials focus on determining how a compound compares to the standard, or most widely accepted, treatment. In Phase III trials, patients are randomly assigned to one of two or more “arms”. In a trial with two arms, for example, one arm will receive the standard treatment (control group) and the other arm will receive treatment with the antisense oligonucleotide or combination of the present invention (investigational group).

Phase IV trials are used to further evaluate the long-term safety and effectiveness of a compound. Phase IV trials are less common than Phase I, II and III trials and will take place after the antisense oligonucleotide or combination has been approved for standard use.

Eligibility of Patients for Clinical Trials

Participant eligibility criteria can range from general (for example, age, sex, type of cancer) to specific (for example, type and number of prior treatments, tumour characteristics, blood cell counts, organ function). Eligibility criteria may also vary with trial phase. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I cancer trials usually comprise 15 to 30 participants for whom other treatment options have not been effective. Phase II trials typically comprise up to 100 participants who have already received chemotherapy, surgery, or radiation treatment, but for whom the treatment has not been effective. Participation in Phase II trials is often restricted based on the previous treatment received. Phase III trials usually comprise hundreds to thousands of participants. This large number of participants is necessary in order to determine whether there are true differences between the effectiveness of the antisense oligonucleotides or combination of the present invention and the standard treatment. Phase III may comprise patients ranging from those newly diagnosed with cancer to those with extensive disease in order to cover the disease continuum.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

Assessment of Patients Prior to Treatment

Prior to commencement of the study, several measures known in the art can be used to first classify the patients. Patients can first be assessed, for example, using the Eastern Cooperative Oncology Group (ECOG) Performance Status (PS) scale or the Karnofsky Performance Status (KPS) scale, both of which are widely accepted standards for the assessment of the progression of a patient's disease as measured by functional impairment in the patient.

Patients' overall quality of life can be assessed, for example, using the McGill Quality of Life Questionnaire (MQOL) (Cohen et al (1995) Palliative Medicine 9: 207-219). The MQOL measures physical symptoms; physical, psychological and existential well-being; support; and overall quality of life. To assess symptoms such as nausea, mood, appetite, insomnia, mobility and fatigue the Symptom Distress Scale (SDS) developed by McCorkle and Young ((1978) Cancer Nursing 1: 373-378) can be used.

Patients can also be classified according to the type and/or stage of their disease and/or by tumour size.

Administration of the Antisense Oligonucleotides Alone or Combinations of the Present Invention in Clinical Trials

The antisense oligonucleotide and the one or more chemotherapeutic agent(s) are typically administered to the trial participants parenterally. In one embodiment, the antisense oligonucleotide or combination is administered by intravenous infusion. Methods of administering drugs by intravenous infusion are known in the art. Usually intravenous infusion takes place over a certain time period, for example, over the course of 60 minutes. In another embodiment, the antisense oligonucleotide is administered to the patient by continuous intravenous infusion.

Monitoring of Patient Outcome

The endpoint of a clinical trial is a measurable outcome that indicates the effectiveness of a treatment under evaluation. The endpoint is established prior to the commencement of the trial and will vary depending on the type and phase of the clinical trial. Examples of endpoints include, for example, tumour response rate—the proportion of trial participants whose tumour was reduced in size by a specific amount, usually described as a percentage; disease-free survival—the amount of time a participant survives without cancer occurring or recurring, usually measured in months; overall survival—the amount of time a participant lives, typically measured from the beginning of the clinical trial until the time of death. For advanced and/or metastatic cancers, disease stabilisation—the proportion of trial participants whose disease has stabilised, for example, whose tumour(s) has ceased to grow and/or metastasise, can be used as an endpoint. Other endpoints include toxicity and quality of life.

Tumour response rate is a typical endpoint in Phase II trials. However, even if a treatment reduces the size of a participant's tumour and lengthens the period of disease-free survival, it may not lengthen overall survival. In such a case, side effects and failure to extend overall survival might outweigh the benefit of longer disease-free survival. Alternatively, the participant's improved quality of life during the tumour-free interval might outweigh other factors. Thus, because tumour response rates are often temporary and may not translate into long-term survival benefits for the participant, response rate is a reasonable measure of a treatment's effectiveness in a Phase II trial whereas participant survival and quality of life are typically used as endpoints in a Phase III trial.

Pharmaceutical Kits

The present invention additionally provides for therapeutic kits containing the antisense oligonucleotide and optionally one or more chemotherapeutic agents in pharmaceutical compositions for use in the treatment of cancer. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a patient.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or any such medically approved delivery vehicle.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

To gain a better understanding of the invention described herein, the following examples are set forth. It should be understood that these examples are for illustrative purposes only. Therefore, they should not limit the scope of this invention in any way.

### EXAMPLES

### Example 1

### Pharmacokinetics and Metabolism in Animals

The pharmacokinetics (PK) of SEQ ID NO:1 (and related oligonucleotide metabolites) were determined in rats and monkeys after single intravenous bolus injections of SEQ ID NO:1 at escalating doses. In addition, the toxicokinetics and/or tissue distribution of SEQ ID NO:1 (and related metabolites) were determined as part of acute (24-hour) and repeat dose (14- and/or 21-days) continuous intravenous infusion toxicity studies in both rats and monkeys. The plasma and tissue analyses were conducted by an appropriately validated (and cross-validated) capillary electrophoresis (CE) method.

1.1 Absorption Pharmacokinetics in the Rat

Groups of Sprague-Dawley rats were administered single intravenous bolus injections of the SEQ ID NO:1 at doses of 10, 25 and 50 mg/kg (59, 147.5, and 295 mg/m²). In each dose group, blood samples were collected from the animals (2 rats/sex/timepoint) at 5, 10, 20, 30, 45 min, and 2, 4, 8 and 24 h post dose. The plasma was prepared for each sample for determination of SEQ ID NO:1 (and metabolites n+1 and n−1 to n−8) concentration.

SEQ ID NO:1 and metabolites were measurable in plasma of the animals in each dose group up to 24 h post dose. Based on AUC and Cₘₐₓ parameters, the plasma levels of SEQ ID NO:1 and its metabolites increased in proportion to administered dose. For SEQ ID NO:1, Cₘₐₓ values were achieved at the first sampling time (5 min) post dose while the maximum metabolite concentrations appeared as a plateau ranging from 5 to 10 min post dose. The elimination of SEQ ID NO:1 from plasma was biphasic with an initial rapid distribution phase followed by a more prolonged apparent terminal elimination phase (ty_{1/2}, 4.72 to 5.80 h). The plasma clearance of SEQ ID NO:1 ranged from 49.67 to 43.30 mL/kg·h. The longer elimination t_{1/2} with reduced plasma clearance tended to occur at the higher dose, suggesting that the elimination pathways may be saturated at that dose level in the rat.

The pharmacokinetics of SEQ ID NO:1 (and metabolites) were also determined as part of a repeat dose toxicity study in rats. For this portion of the study, 8 rats/sex were assigned as satellite animals and administered SEQ ID NO:1 at a dose of 50 mg/kg/day (295 mg/m²) (reduced to 40 mg/kg/day (236 mg/m²) by continuous intravenous infusion for 14 days. The concentrations of SEQ ID NO:1 (and metabolites n+1 and n−1 through n−8) were measured in the plasma, with the results of the plasma analyses used for determination of pharmacokinetic parameters.

Based on the plasma SEQ ID NO:1 (and metabolites) concentrations during infusion, there were no apparent differences in the levels between males and females. Based on the mean plasma concentrations during infusion, steady state levels (Cₛₛ) were achieved after approximately 24 h of continuous infusion: SEQ ID NO:1, 34.8 μg/mL; metabolites, 64.2 μg/mL. Elimination t_{1/2}, for SEQ ID NO:1 was calculated to be 8.6 h. The plasma clearance (Cl), calculated on the basis of Cₛₛ and infusion rate was 47.8 mL/kg·h. The mean Cₘₐₓ values for SEQ ID NO:1 and SEQ ID NO:1 metabolites (n+1 and n−1 to n−8) were 40.4 and 82.2 μg/mL, respectively. The median time at which Cₘₐₓ occurred was 96 h for both SEQ ID NO:1 and SEQ ID NO:1 metabolites.

1.2 Pharmacokinetics in the Monkey

Two groups of Cynomolgus monkeys were administered single intravenous injections of SEQ ID NO:1 as doses of 10 mg/kg (123 mg/m²) and 50 mg/kg (615 mg/rn²). Serial blood samples were withdrawn from each animal at 0 (prior to dosing), 10, 20, 60, 90 min and 2, 3, 6, 8 and 24 h post injection. Plasma concentrations of SEQ ID NO:1 (and metabolites, n+1 and n−1 to n−8) were determined, and the results of the plasma levels were used for determination of the pharmacokinetic parameters.

Based on non-compartmental analysis, the plasma elimination of SEQ ID NO:1 was determined to be biphasic in each treatment group. For each monkey, the Cₘₐₓ and AUC estimates were proportional with the administered dose for both SEQ ID NO:1 and its metabolites. Tₘₐₓ (observed) of SEQ ID NO:1 and metabolites was generally recorded at the first blood sampling timepoint (10 min post dosing) in all animals, except one male in the high dose group where the Tₘₐₓ (observed) for metabolites was recorded at the second blood sampling timepoint (20 min post dosing). There was a slightly increased mean Cl value and significantly larger mean elimination t_{1/2} for the high dose group, compared to the low dose group. There were no clear observed sex differences in the pharmacokinetics of SEQ ID NO:1.

The toxicokinetics of SEQ ID NO:1 (and metabolites) were also determined as part of the repeat dose (14-day and 21-day) toxicity studies in monkeys. In the repeat dose study, groups of male and female monkeys were administered SEQ ID NO:1 by continuous intravenous infusion for 14-days (Part 1) and 21-days (Part 2) at dose levels of 10, 20 and 40 mg/kg/day (123, 246, and 492 mg/m²/day) (Part 1) and 2, 10 and 50 mg/kg/day (24.6, 123, and 615 mg/m²/day) (Part 2). In Part 1, serial blood samples were withdrawn from each animal at 0 (pretreatment), 8, 48, 168 and 336 h following the onset of infusion, and from the recovery animal (male, high dose only) at 20, 60, 90 and 180 min post end of infusion. In Part 2, serial blood samples were withdrawn from each animal at 0 (pretreatment), 8, 24, 48, 96, 168, 336 and 480 h following the onset of infusion, and from the recovery animals (1/sex, high dose only) at 20, 60, 90 and 180 min post end of infusion.

Based on the results in both Part 1 and 2, there were no apparent sex-differences in the toxicokinetic profile of SEQ ID NO:1 and its metabolites. In both Parts 1 and 2, the time to Css for the test article was consistently achieved at the first or second blood collection timepoint in all treatment groups. The estimates of elimination t_{1/2} in the recovery monkeys were found to be consistent between the one Part 1 monkey, and the two Part 2 monkeys, ranging from 2.2 to 2.5 hours. However, considerable interindividual variability was found for the plasma Cl of SEQ ID NO:1, where values ranged from 45.7 to 116.7 mL/kg·h, with no apparent correlation to dose level or duration of infusion. AUC estimates, for both SEQ ID NO:1 and its summed metabolites, were proportional with the duration of infusion and the administered dose level in the Part 1 and Part 2 Recovery animals.

1.3 Tissue Distribution

The tissue distribution of SEQ ID NO:1 (and metabolites) was determined in rats and monkeys as part of the repeat dose toxicity studies in those species. In general, following continuous infusion, the distribution of SEQ ID NO:1 (and metabolites) in both rats and monkeys was consistent with observations reported for other phosphorothioate oligonucleotides. The highest concentrations of SEQ ID NO:1 (and metabolites) were observed in the kidney>liver>spleen>lymph node (monkey)>lung (monkey)>heart. The levels in the brain were very low or below the limits of detection in both species suggesting that SEQ ID NO:1 (and metabolites) did not significantly cross the blood brain barrier.

The results of the repeat dose toxicity evaluations indicated that in both species the tissues manifesting the histopathological abnormalities included the kidney, liver, and lymph nodes. Since the highest concentrations of SEQ ID NO:1 and metabolites were found in those tissues, these data suggest that there was a relationship between the concentration of parent oligonucleotide (and metabolites) with morphological and with functional changes in those tissues. Upon discontinuation of SEQ ID NO:1 treatment, there was evidence in monkeys that both the parent compound and its metabolite levels in various tissues decreased over time.

1.4Metabolism

The principal metabolic pathway for oligonucleotides is cleavage via endo- and exonucleases (Cossum et al., 1993; Cossum et al., 1994; Iversen, 1991). Metabolism mediated by exo- and endonucleases results in shorter oligonucleotides and, ultimately, nucleosides that are degraded by normal metabolic pathways. The pattern of metabolites suggests primarily exonuclease activity with perhaps modest contributions by endonucleases.

1.5 Excretion

Phosphorothioate oligonucleotides are primarily eliminated in urine, with as much as 40% eliminated in 24 hours and up to 70% eliminated in 240 hours (Agrawal 1991; Zhang 1995; Iverson 1991; Srinivasan 1995; Grindel 1998). Fecal excretion is a minor pathway of elimination (Agrawal 1991; Zhang 1995). Oligonucleotides are excreted in urine mainly in a degraded form, although some intact oligonucleotide has been detected in urine at higher doses (≧30 mg/kg) (Agrawal 1991).

### Example 2

### Toxicology Studies

2.1 Single Dose Toxicity

2.1.1 Acute Intravenous Toxicity Study of SEQ ID NO:1 in Rats

The purpose of this study was to assess the adverse effects of SEQ ID NO:1 when administered as a single intravenous dose to Sprague-Dawley rats. In this study, four groups of animals (3/sex/group) were administered SEQ ID NO:1 by continuous intravenous infusion for 24-hours at escalating doses. Subsequent dose levels were incrementally escalated as follows when toxicological effects were not observed at the 40 mg/kg/day dose: 60, 80 and 90 mg/kg. Parameters assessed included mortality, clinical observations, body weight and food consumption assessment, clinical pathology and urinalysis measurements, and gross examination at necropsy.

The results indicated some test article related effects were found in animals that received doses of 60, 80 and 90 mg/kg.

2.1.2 Acute Intravenous Toxicity Study of SEQ ID NO:1 in the Monkey

The objective of this single, dose escalating study was to establish a maximum tolerated dose (MTD) for SEQ ID NO:1 and to assess the effect of administered SEQ ID NO:1 on the cardiac function of conscious Cynomolgus monkeys. In this study, three monkeys (two males and one female) were administered SEQ ID NO:1 by continuous intravenous infusion for 24-hours at escalating doses of 10, 20, 40 and 80 mg/kg. There was a 3-day washout between doses. One female animal, administered vehicle only (PBS) in the same manner, was used as a control. Electrocardiogram (ECG) measurements were conducted on each animal (including control) prior to, during and after the end of each infusion interval. Clinical signs, mortality, body weight and food consumption measurements, hematology, coagulation and clinical chemistry parameter evaluations, were recorded and evaluated. In addition, blood samples were removed prior to and at the end of each infusion interval to measure complement (CH50 and Bb). Blood samples were also drawn at the end of each infusion for analysis of parent, SEQ ID NO:1.

There were no deaths, clinical signs, body weight changes, or effects on food consumption. There were no treatment-related effects on ECG recordings and blood pressure. There was an apparent increasing trend in activated partial thromboplastin times (APTI) following the last dose (Day 14) in all animals; however, all values were within normal ranges. The complement analyses indicated that up to 40 mg/kg, the Bb and CH50 values were within the reference range. At the high dose (80 mg/kg), the complement values from one animal (female) were outside the normal range (higher Bb and lower CH50) suggesting that complement activation had occurred. Analysis of plasma samples obtained at the end of infusion indicated that the concentrations of SEQ ID NO:1 increased with escalating dose levels. These effects indicate an apparent inhibition of the intrinsic coagulation pathway and modest actuation of the alternative complement pathway response. These treatment-related changes were typical class effects of phosphorothioate oligonucleotide administration.

2.2 Repeat Dose Toxicity

2.2.1 A 14-Day Continuous Intravenous Infusions Toxicity Study of SEQ ID NO:1 in the Rat with a 14-Day Recovery

The objective of this study was to assess the potential adverse effects of SEQ ID NO:1 in male and female Sprague Dawley rats when administered by continuous intravenous infusion for 14 days. Ten rats/sex/group were administered SEQ ID NO:1 at doses of 0 (control), 2, 10, or 50 mg/kg/day. Animals in the control group received the vehicle article, PBS. However, due to severe adverse clinical signs and mortality of animals in the 50 mg/kg/day dose group, the high dose was reduced to 40 mg/kg/day on days 8, 9, and 10. An additional 5 rats/group were included in the control and high dose group as recovery animals, and were allowed a 14-day observation period following the treatment period. Parameters assessed during the study include mortality, clinical signs, body weights, food consumption, opthalmoscopic examination, clinical pathology assessment (hematology, coagulation, clinical chemistry, and urinalysis). Terminal procedures included a complete necropsy of each animal, and histopathologic evaluation of selected tissues for animals in the control and high dose groups. For toxicokinetic evaluations, an additional 8 rats/sex were included in the high dose groups. Serial and terminal blood samples were withdrawn from satellite animals at selected time points during infusion and after the end of infusion. Designated tissues were also collected from selected toxicokinetic animals and analyzed for SEQ ID NO:1 (and metabolites) concentration.

Treatment-related effects were found in the high dose group, including high morbidity and mortality, reduced body weights and reduced food consumption. Clinical pathology results showed dose-dependent anemia, thrombocytopenia, coagulopathic selectivity for APTT, and liver and kidney toxicity in both sexes. Pathological findings strongly correlated with these results, and showed major treatment-related changes in numerous tissues and organs of the high dose animals, in most tissues and organs of the mid dose animals; and sporadically in the evaluated tissues and organs of the low dose animals. The adverse effects to SEQ ID NO:1 treatment appeared more pronounced in males than females suggestive of an apparent sex effect. The toxicokinetic results from animals infused at the high dose level indicated that SEQ ID NO:1 CS, achieved at approximately 24 h after start of infusion, was 34.8 μg/mL. The apparent t % was 8.6 h and the total plasma clearance was 46.9 mL/kg·h. Tissue uptake of SEQ ID NO:1 (and metabolites) was highest in the kidney followed by the liver, spleen and heart. The levels in the brain were undetectable. This pattern of tissue distribution was considered typical of phosphorothioate oligonucleotides. Many of the adverse effects that were found in the animals of this study appeared to correlate to the high levels of SEQ ID NO:1 (and metabolites) in the kidney, liver and spleen.

2.2.2 Repeat Dose (14- and 21-day) Toxicity Study in Monkeys with a 14 or 21-day Recovery Period

The objective of this study was to assess the toxicity and toxicokinetics of SEQ ID NO:1 in male and female Cynomolgus monkeys after continuous intravenous infusion for 14 (Part 1) or 21 (Part 2) days. The reversibility of potential toxic effects of SEQ ID NO:1 at the highest dose level was also assessed during, a 14 or 21-day recovery period. In the 14-day study (Part 1), groups of one male and one female monkey recovered daily doses of 0 (vehicle, PBS), 10, 20 or 40 mg/kg of SEQ ID NO:1 by continuous intravenous infusion for 14 consecutive days. An additional male was assigned to each of the vehicle and high dose group and remained on study for 14-day recovery period. In the 21-day study (Part 2), four groups of three male and three female monkeys received daily doses of 0 (vehicle, PBS), 2, 10, or 50 mg/kg of SEQ ID NO:1 by continuous intravenous infusion for 21 consecutive days. An additional male and female were assigned to each of the control and high dose groups, and remained on study for 21 days after conclusion of dosing. Parameters evaluated for Part 1 and 2 included clinical signs; body weights, food consumption, appetite, clinical pathology assessment (hematology, clinical chemistry, coagulation, and urinalysis), ECG assessment, opthalmoscopy examinations, and immunology measurements (complement split products Bb analysis). Blood and tissue samples (at necropsy) were collected and analyzed for SEQ ID NO:1 (and metabolites) concentration. At termination, surviving animals were euthanized and subjected to macroscopic and microscopic examination.

In Part 1 (14-day infusion up to 40 mg/kg/day), there were no deaths, no treatment-related clinical signs, effects on appetite, ophthalmology effects, cardiology, hematology effects, or changes in organ weights. There was a slight decrease in body weight in one mid and high male, however, it was unclear if it was related to SEQ ID NO:1 administration. A reversible increase in activated partial thromboplastin time (APTT) was found in the high dose animals on Day 14. Bb appeared to increase in the high dose group on Day 14. Transient changes in some clinical parameters were noted in mid and low dose animals but were not considered toxicologically significant. Treatment-related macroscopic and histopathological changes were noted in the liver, kidneys, lymph nodes, infusion sites, and adrenals (high dose only). In the recovery animal, similar but less severe effects were noted in the kidneys, lymph nodes and infusion site.

In Part 2, one high dose animal was sacrificed on Day 19 for ethical reasons. Treatment-related reversible clinical signs were limited to a few high dose animals, mainly males, and were reflective of weakness (e.g., decreased activity, decrease appetite, pallor, cold to touch). A slight decrease in body weight was observed in some high dose males; however, at the end of the recovery period both high dose animals had gained weights. There was a significant decrease in appetite noted in the majority of high dose males, in addition to a slight decrease in one high dose female. Bilateral retinopathy was noted in one high dose male at the end of the treatment period, but due to the low incidence, the significance of this finding is unclear. A marked increase in WBC counts was observed on Day 20 in all high dose animals, which was associated with high neutrophil, monocyte, and/or large unstained cell counts. In addition, slight to moderate reductions in red blood cell, hematocrit, and hemoglobin values, suggestive of anemia were noted in three high dose animals, along with one animal in the mid and low dose groups in addition to two control animals. APTT values were significantly longer on Day 20 in animals from the high dose group, compared to control values. Changes in organ weights were limited to a slight increase in relative and absolute kidney weights, which were noted in two high dose females along with the recovery male. Treatment-related histopathology changes were noted in the liver, kidneys, adrenals (high dose only), lymph nodes (mid and high-dose), brain (high dose only), heart (high-dose only), thymus, and infusion sites. In the kidneys, the severity of findings was dose-related. In the recovery animals, similar but less severe changes were noted in the kidneys, liver, and lymph nodes. In addition minimal to moderate lymphoid atrophy was noted in all groups, including the control and recovery animals. Because of its increased incidence and severity in the high dose group, this finding was considered to be an indirect effect of SEQ ID NO:1.

In summary, the administration of SEQ ID NO:1 for 14 days at 10, 20 or 40 mg/kg/day produced partially reversible treatment-related effects, that were limited to prolongation of activated partial thromboplastin time (40 mg/kg/day), and microscopic changes in various tissues and organs (all groups). Administration of SEQ ID NO:1 for 21 days at 50 mg/kg/day was associated with reversible signs of weakness, decreases in body weight and appetite, prolongation of APTT, anemia, thrombocytopenia, and monocytosis. This dose level also resulted in increased kidney weight, along with microscopic changes in various organs that were partially reversible after a 21-day recovery period. Treatment-related changes at 2 and 10 mg/kg/day were limited to slight anemia and multiorgan microscopic changes. Most of the treatment related effects noted were similar and consistent with those observed in monkey studies for compounds of the same chemical class.

2.3 In Vitro Hemolysis

SEQ ID NO:1 injection was tested for its potential to cause hemolytic activity based on cell lysis and hemoglobin release in human whole blood. Four milliliters of each concentration of dosing solution (1.0, 5.0, and 10 mg/mL), 0.99% saline (negative control), or 1% saponin (positive control) were mixed with 5.0 mL of diluted blood and incubated for one hour at 37±1° C. under static and dynamic conditions. Following the test procedures, the hemolytic index was calculated. The test article was non-hemolytic under both static and dynamic conditions. None of the test article concentrations had a hemolytic index of greater than 2.

### Example 3

### Effects of SEQ ID NO:10N PC-3 Prostate Tumor Growth in SCID Mice

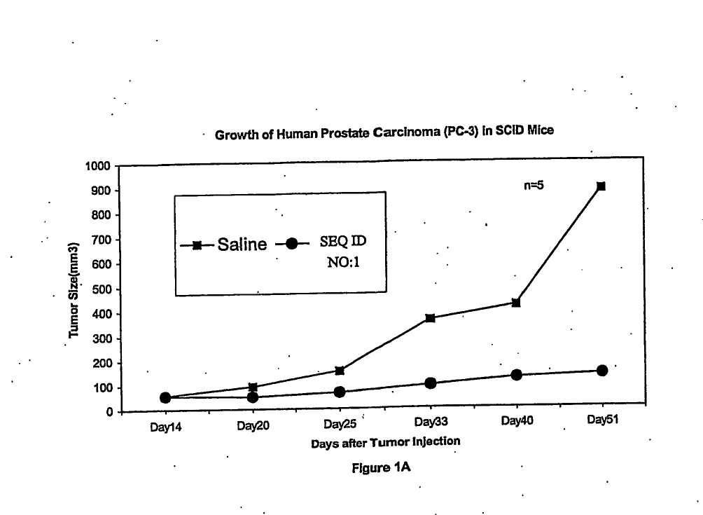

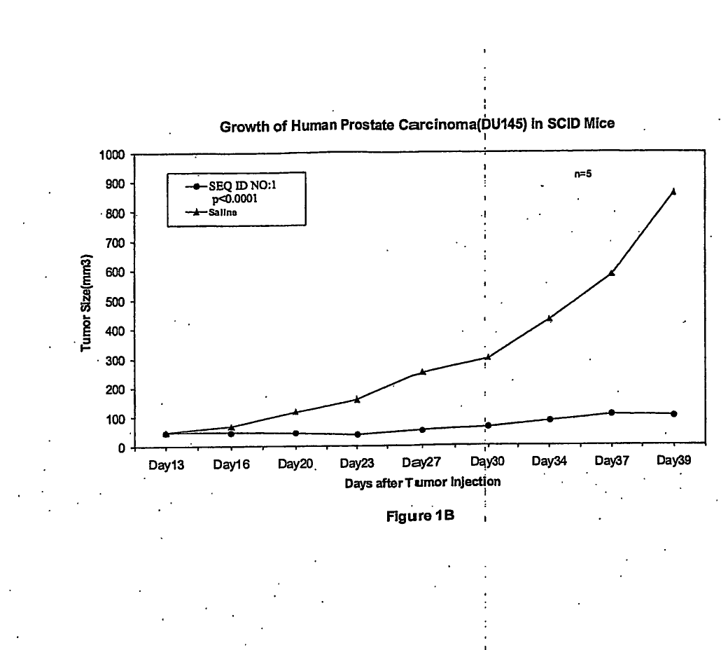

PC-3 human prostatic cancer cells (1×10⁷ cells in 100 μl of PBS) were subcutaneously injected into the right flank of 6-7 week old male SCID mice. After the tumor size reached an approximate volume of 50 mm³, 14 days post tumor cell injection, SEQ ID NO:1 was administered by bolus infusion into the tail vein every other day at 10 mg/kg. Control animals received saline alone for the same period. Treatments lasted for 36 days thereafter. Antitumor activities were estimated by the inhibition of tumor volume, which was measured with a caliper on six different occasions over 36-day period. Each point represents mean tumor volume calculated from 5 animals per experimental group. As illustrated in FIG. 1A, SEQ ID NO:1 treatment demonstrated strong inhibitory effects on the growth of human prostate carcinoma.

DU145 human prostatic cancer cells (1×10⁷ cells in 100 μl of PBS) were subcutaneously injected into the right flank of 6-7 week old male SCID mice. After the size of the tumors reached an approximate volume of 50 mm³, 13 days post tumor cell injection, SEQ ID NO:1 was administered by bolus infusion into the tail vein every other day at 10 mg/kg. Control animals received saline alone for the same period. Treatments lasted for 30 days thereafter. Antitumor activities were estimated by the inhibition of tumor volume, which was measured with a caliper on nine different occasions over 30-day period. Each point represents mean tumor volume calculated from 5 animals per experimental group. As illustrated in FIG. 1B, SEQ ID NO:1 treatment demonstrated strong inhibitory effects on the growth of human prostate carcinoma.

### Example 4

### Effects of Combination Therapy on Prostate Tumor Growth in SCID Mice

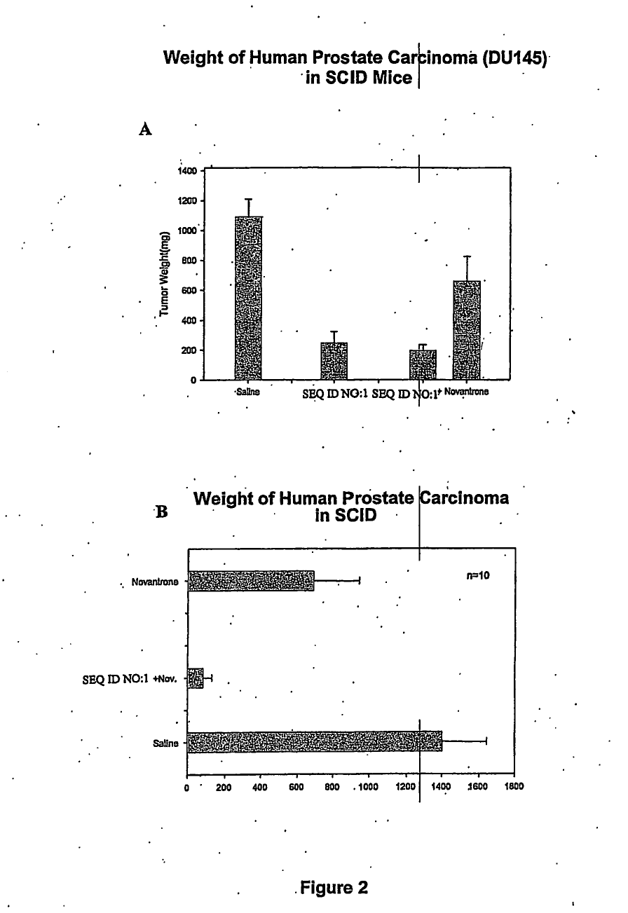

FIG. 2 shows results from two independent experiments. In both experiments, DU145 human prostatic cancer cells (1×10⁷ cells in 100 μl of PBS) were subcutaneously injected into the right flank of 6-7 weeks old male SCID mice. After the size of tumor reached an approximate volume of 50 mm³, 13 (upper panel) or 11 (lower panel) days post tumor cell injection, SEQ ID NO:1 was administered by bolus infusion into the tail vein every other day at 10 mg/kg 15 times (upper panel) or 14 times (lower panel), respectively. Control animals received saline alone for the same period. Antitumor effect of SEQ ID NO:1 was further compared to that of mitoxantrone (novantrone®) alone or in combination. Mitoxantrone was administered intravenously once at the beginning of the treatments at a dose of 2 mg/kg (upper panel) or once a week for four weeks at a reduced dose of 0.8 mg/kg (lower panel). All treatments were stopped at day 42 (upper panel) or 38 (lower panel), respectively. A day after the last treatment, tumors were excised from the animals and their weights were measured. A standard bar graph (FIG. 2) was used to demonstrate the differences in tumor weights with each bar representing mean tumor weight calculated from 5 (upper panel) or 10 (lower panel) animals. As illustrated in the left panel, SEQ ID NO:1 treatments resulted in significant delay of tumor growth compared to saline control. The delay in tumor growth achieved with SEQ ID NO:1 was superior to the inhibitory effects observed with mitoxantrone alone. The combination of SEQ ID NO:1 with mitoxantrone (SEQ ID NO:1+) showed some additive antitumor effects. In the lower panel, mitoxantrone alone resulted in delay of tumor growth and the combination therapy was significantly more potent than mitoxantrone monotherapy.

### Example 5

### Immune Related Issues

An issue that must inevitably be addressed when developing antisense therapeutics is whether the compound produces non-specific immune stimulation that is not a result of target sequence interactions. Immune stimulation can be the result of two properties of AS-ODN, one sequence specific and one backbone specific. Un-methylated CpG di-nucleotides, usually present in bacterial DNA, stimulate innate immune responses in vertebrates and can further augment acquired immune responses to both pathogens and tumor cells. The presence of un-methylated CpGs in an oligonucleotide can have the same effect if in an optimal sequence context. In addition, the phosphorothioate backbone, used in first generation antisense compounds, has been found to be immune stimulatory in a sequence independent manner. As shown in a number of tumor xenograft experiments, SEQ ID NO:1 is highly effective in SCID mice that are T and B cell deficient suggesting SEQ ID NO:1 acts independent of the acquired immune system. There is strong evidence from other studies that NK cells are stimulated by CpG motifs.

5.1 Effects of SEQ ID NO:1 on Caki Renal Tumor Growth in SCID/Beige Mice

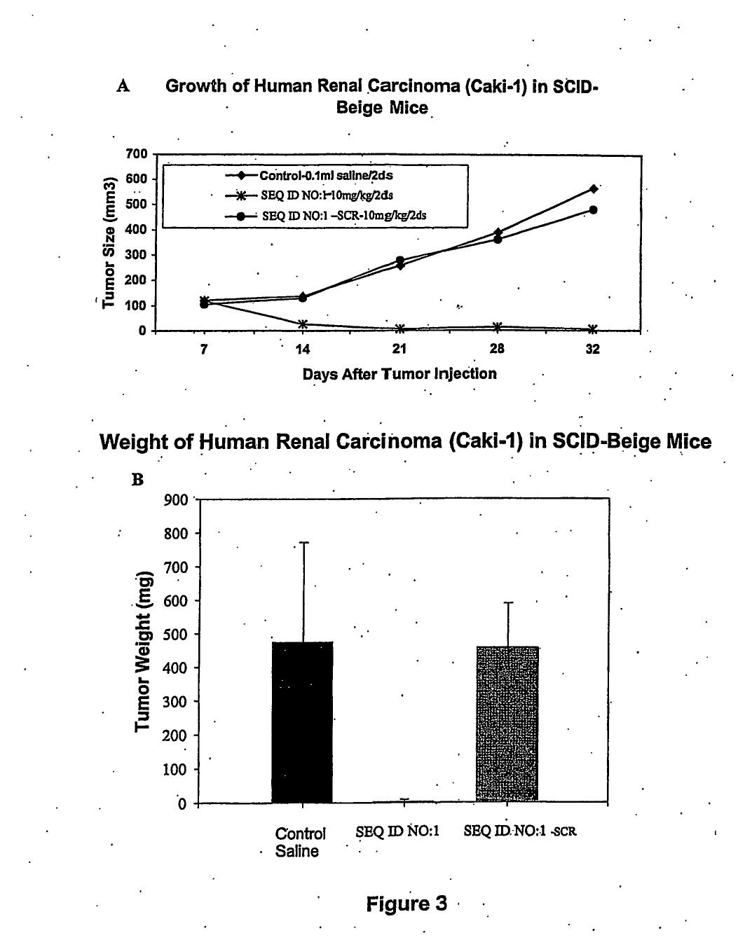

To address whether SEQ ID NO:1 anti-tumor activity is NK mediated, tumor xenograft growth was assessed in SCID/beige mice that are NK, T and B cell deficient. Caki-1 human kidney cancer cells (5×10⁶ cells in 100 μl of PBS) were subcutaneously injected into the right flank of 6-7 weeks old female SCID/beige mice. After the size of tumor reached an approximate volume of 100 mm³, 7 days post tumor cell injection, SEQ ID NO:1 and SEQ ID NO:1-SCR were administered (10 mg/kg/2 days, i.v.). Control animals received saline alone for the same period. Caliper measurements at 1 week intervals were used to calculate tumor volumes. Each point in FIG. 3 (top) represents mean tumor volume calculated from 10 animals per experimental group. After 32 days the mice were sacrificed and the tumors weighed. Each bar in FIG. 3 (bottom) represents the mean tumor weight and standard error calculated for each treatment group. SEQ ID NO:1 was highly effective against renal tumor xenografts in these mice. Other studies have demonstrated that the anti-tumor efficacy of immuno-stimulatory CpG ODNs is compromised in murine tumor models using these mice, consistent with SEQ ID NO:1 not acting via immune stimulation.

### Example 6

### Inhibition of R1 mRNA in Tumors

Effects of SEQ ID NO:1 administration on R1 mRNA levels in HT-29 colon tumors in nude mice were investigated.

Methods.

For determination of mRNA levels in tumors by Northern Blot, total RNA was prepared from excised tumors using TRIzol reagent (GIBCO BRL). Northern blot analysis was performed as previously described (Hurta and Wright, 1995). RNA was subjected to electrophoresis through 1% formaldehyde agarose gels followed by transfer to nylon membranes. Blots were hybridized in the presence of a R1 fragment (McClarty et al, 1987). Glyceraldehyde-3-phosphate dehydrogenase (GAPDH) mRNA levels were simultaneously probed for RNA loading controls.

Results.

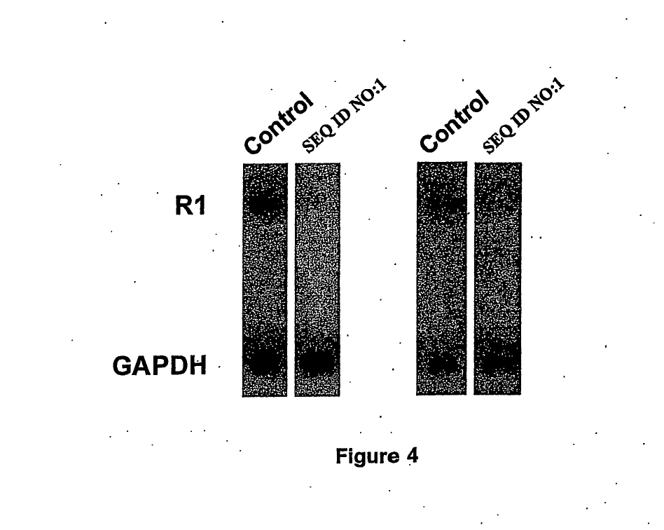

As shown in FIG. 4, marked reduction in the R1 mRNA levels was observed in two independent HT-29 tumors at day 16 following administration of SEQ ID NO:1 every other day at a dose of 10 mg/kg. The results provide strong evidence that SEQ ID NO:1 is reaching the tumor site in vivo and is acting by an antisense mechanism of action.

SEQ ID NO:1 decreased R1 mRNA levels in HT-29 colon tumors xenografted into mice (FIG. 4). Tumors of sufficient size were not obtainable for many tumor types and use of surrogate mouse tissue was not appropriate due to target sequence differences.

### Example 7

### Expression of R1 in Normal and Tumor Cell Lines

Methods.

To measure R1 protein levels, western blot analysis was conducted. Briefly, cells were washed once with PBS and whole cell protein extracts were prepared in 50-150 μl of 2× sample loading buffer (100 mM Tris, pH 6.8, 200 mM DTT, 4% SDS, 20% glycerol and 0.015% bromphenol blue). Extracted protein (10-20 μg) was fractionated on 12% SDS-PAGE, transferred to nitrocellulose membranes and total protein visualized by India ink staining. R1 protein was detected with AD 203, an anti-R1-antibody (5-50 μg/ml; obtained from either InRo BIOMEDTEK, Sweden or Accurate Chemical and Scientific Corporation, Westbury, N.Y., USA) followed by horseradish peroxidase-conjugated goat anti-rabbit IgG (Sigma, St. Louis, Mo.) at a dilution of 1:5,000. The 80 kDa R1 protein was visualized by development of the peroxidase reaction (ECL chemiluminescence, Amersham Corporation). GAPDH protein was detected as an internal control. WI-38 and HUVEC cells are normal cell lines. The remainder are tumor cell lines routinely used in xenograft tumor model studies.

Results.

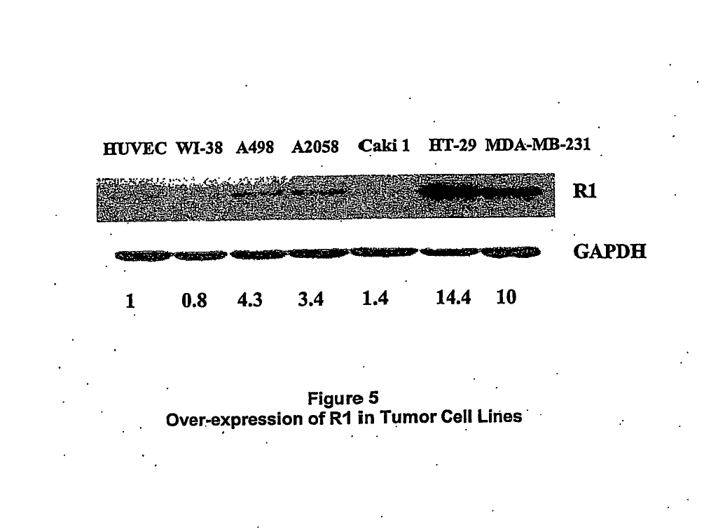

Earlier studies have demonstrated elevated RNR levels and activity in tumors and tumor cell lines. To assess whether this is a general phenomenon of cancer cells, the R1 protein levels were examined in untreated cancer cell lines derived from diverse cancer types, including renal, skin, colon and breast cancer cell lines (FIG. 5). The R1 expression was compared to R1 expression in 2 normal cell lines, WI38 and HUVEC. GAPDH, protein expression was determined as an internal reference. Consistent with its role in cancer progression, R1 levels were elevated in all of the tumor cell lines tested. The increase in R1 varied from 1.4-14 fold, compared to HUVEC cells, and 1.8-17 fold, compared to WI-38 cells. These data support the targeting of R1 for down-regulation via antisense compounds.

R1 protein is over-expressed in a number of tumor cell lines making R1 a good tumor target (FIG. 5).

### Example 8

### Inhibition of the Growth of Tumor Cell Lines

The effect of SEQ ID NO:1 on the colony forming ability were evaluated in the following human tumor cell lines:

| | |
|---|---|
| Hep G2 (liver) | SK-OV-3 (ovary) |
| U-87 MG (brain) | A2058 (melanoma) |
| H460 (lung) | MDA-MB-231 (breast) |
| AsPC-1 (pancreas). | |

Methods.

Tumor cells were washed in 5 ml of phosphate buffered saline, pH 7.2, prior to 0.2 μM antisense oligonucleotide/lipofectin treatment for 4 hours. The medium was removed and the cells were gently washed with 5 ml of growth medium. The cells were then cultured in growth medium for seven to ten days. Surviving colonies were visualized by methylene blue staining and colonies of 50 or more cells were scored (Choy et al., 1988 and Huang and Wright, 1994). Results are summarized from 4 to 8 trials for each tumor cell line.

Results.

A greater than 60%0 inhibition in colony forming ability was observed for all cell lines treated with SEQ ID NO:1 with the exception of the U-87 MG (brain), MDA-MB-231 (breast) and AsPC-1 (pancreas). A decrease of roughly 40 to 60% in colony forming ability was observed in these three cell lines following administration of antisense oligonucleotide.

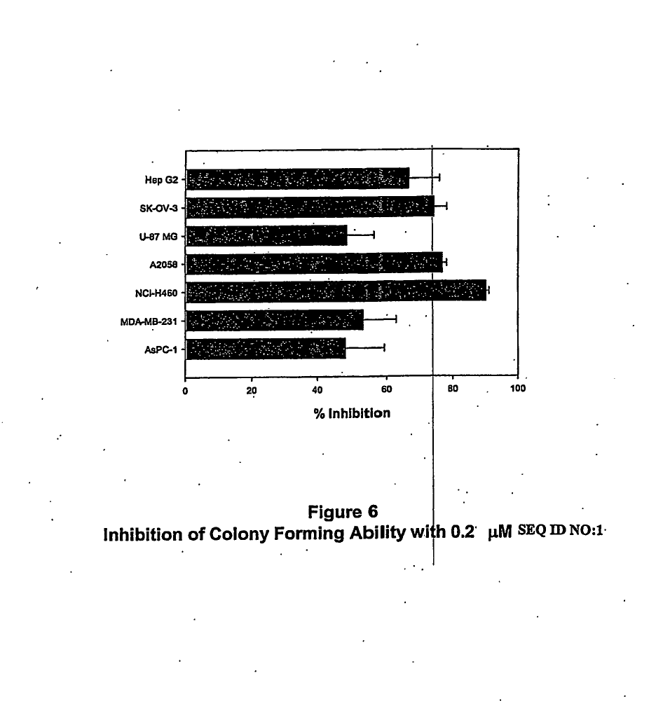

SEQ ID NO:1 inhibited the growth of human tumor cell growth in colony forming assays (FIG. 6).

### Example 9

### Inhibition of the R1 target at the mrna level

Northern blot analyses were performed to determine if SEQ ID NO:1 treatment of human tumor cell lines had an effect on R1 mRNA levels. Results of these assays demonstrate that SEQ ID NO:1 specifically decreases R1 mRNA.

Methods.

Total RNA was isolated using TRIzol reagent (GIBCO BRL) after cells were treated with 0.2 μM SEQ ID NO:1 for four hours in the presence of cationic lipid (Lipofectin reagent, GIBCO BRL), washed with PBS, and incubated for 16 hours to recover in normal medium containing 10% FBS. Northern blot analysis was performed as previously described (Hurta and Wright, 1995). RNA was subjected to electrophoresis through 1% formaldehyde agarose gels followed by transfer to nylon membranes. Blots were hybridized in the presence of a R1 fragment (McClarty et al, 1987). Glyceraldehyde-3-phosphate dehydrogenase (GAPDH) mRNA levels were simultaneously probed for RNA loading controls.

Results.

A significant decrease in R1 mRNA was observed following SEQ ID NO:1 treatment of HT-29 (human colon adenocarcinoma) and MDA-MB-231 human breast adenocarcinoma) cell lines. The results are shown below. Two independent treatments with SEQ ID NO:1 (1 and 2) consistently resulted in marked reduction in the R1 mRNA levels in both cell lines.

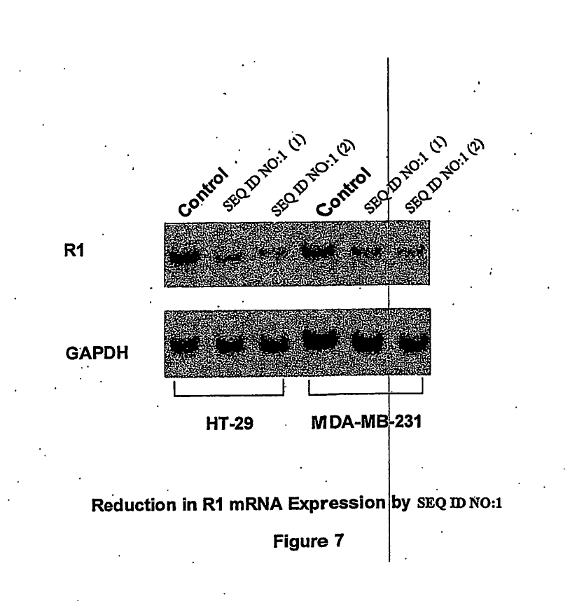

Incubation of 0.2 μM SEQ ID NO:1 with human colon or breast adenocarcinoma cells decreased R1 mRNA levels in those cells (FIG. 7).

### Example 10

### Inhibition of the R1 Target at the Protein Level

10.1 Immunoprecipitation

Immunoprecipitation analyses were performed to determine if SEQ ID NO:1 treatment of human tumor cell lines had an effect on R1 protein expression. Results of these assays demonstrate that SEQ ID NO:1 specifically decreases R1 protein expression.

Methods.

Immunoprecipitation was performed using a saturating amount of AD203 anti-R1 monoclonal antibody as previously described (Choy et al., 1988). Human tumor cells, AsPC-1 (pancreatic adenocarcinoma), were exposed for 4 hours to SEQ ID NO:1, SEQ ID NO:1 M is (a SEQ ID NO:1 sequence containing four base mismatch) or SEQ ID NO:1 Scr (a sequence with the same ratio of ACTG as the SEQ ID NO:1 sequence but scrambled). Cells were then washed and labeled with ³⁵S-methionine for 4-7 hours. R1 protein was specifically immunoprecipitated with R1 antibody from cell lysate, resolved on sodium dodecyl sulfate-polyacrylamide gels and analyzed by autoradiography.

Results.

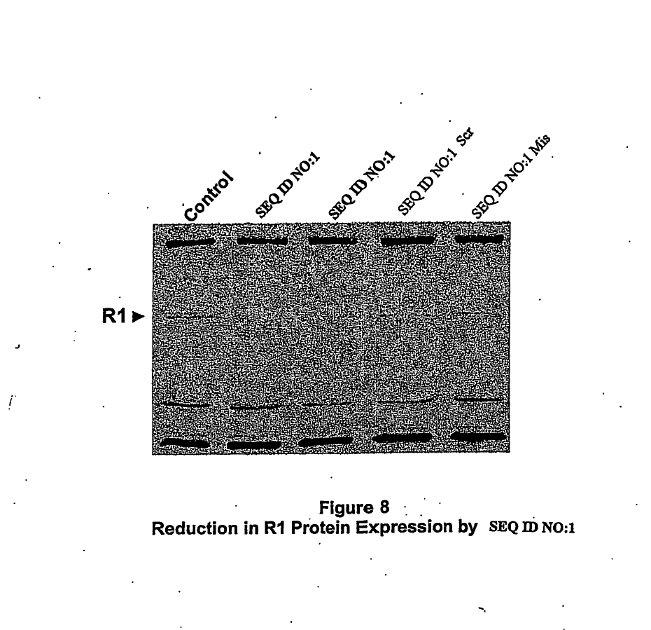

FIG. 8 shows the results. Newly synthesized R1 protein was specifically precipitated with R1 antibody in the cells that were not treated with antisense oligonucleotides (Control). R1 protein expression, however, was dramatically decreased following exposure of tumor cells to 0.2 μM SEQ ID NO:1 (SEQ ID NO:1). There was no significant decrease in R1 protein synthesis following administration of 0.2 μM of either a SEQ ID NO:1 Scr or SEQ ID NO:1 Mis.

Immunoprecipitation analysis using R1 specific antibody to measure the synthesis of R1 protein in the AsPC-1 pancreatic adenocarcinoma cells demonstrated that 0.2 μM SEQ ID NO:1 specifically inhibit the expression of R1 protein. In contrast, incubation with a SEQ ID NO:1 sequence with a four base mismatch or an oligonucleotide with the same ratio of ACTG as SEQ ID NO:1 but scrambled did not decrease R1 protein expression (FIG. 8).

10.2 Western Blots

Western blots were performed to determine if SEQ ID NO:1 treatment of human tumor cell lines had an effect on R1 protein expression. Results of these assays demonstrate that SEQ ID NO:1 specifically decreases R1 protein expression in a dose-dependent manner.

Methods.

MDA-MB-231 human breast adenocarcinoma cells were treated with increasing concentrations (0.025-0.2 μM) of SEQ ID NO:1, 0.2 μM of a scrambled control analogue of SEQ ID NO:1 (SEQ ID NO:1 Scr) or a mismatched control analogue of SEQ ID NO:1 (SEQ ID NO:1 Mis) that contains four base changes. Cells were then washed and fresh media were added. Cells were harvested 8-18 hours later for protein extractions. Aliquots of cell extracts were heated at 100° C. for 5 minutes and then analyzed on sodium dodecyl sulfate-polyacrylamide gels (Choy et al., 1988). Proteins were then transferred to membranes. Membranes were blocked and then incubated with anti-R1 antibody for 1 hour at room temperature. Membranes were washed three times in cold TBS-Tween buffer followed by incubation for 30 minutes to 1 hour at room temperature in the presence of a second antibody (goat anti-rabbit immunoglobulin linked with horseradish peroxidase). Blots were washed and bound antibodies were detected by development of the alkaline phosphatase reaction (Fan et al., 1996).

Results.

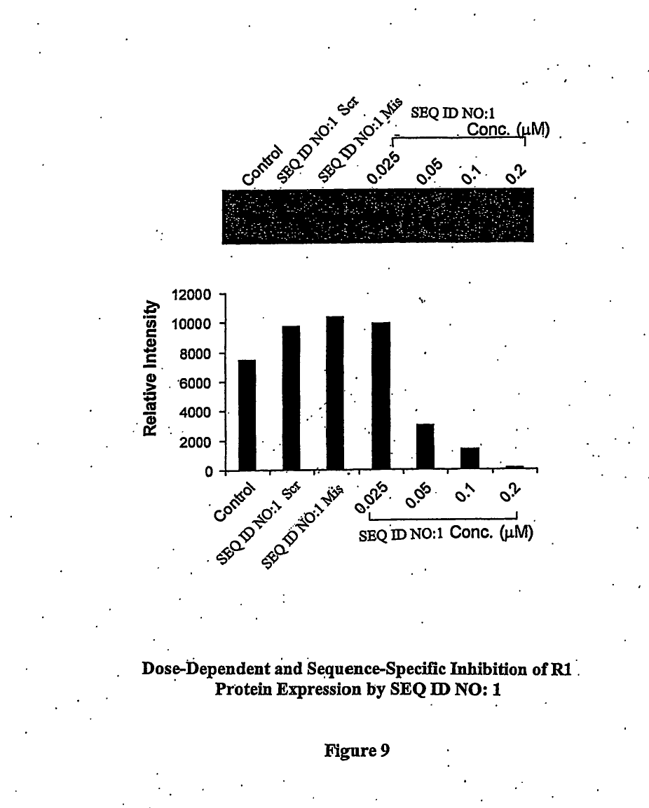

The results in FIG. 9 show that R1 protein expression decreased in a dose-dependent manner following exposure of tumor cells to increasing concentrations of SEQ ID NO:1. There was no decrease in R1 protein following the administration of 0.2 μM of either a scrambled version of SEQ ID NO:1 (SEQ ID NO:1 Scr) or a four base pair mismatch of SEQ ID NO:1 (SEQ ID NO:1 Mis). Densitometric measurements of each band are expressed as a relative intensity as illustrated below.

Incubation of increasing concentrations of SEQ ID NO:1 (0.025 to 0.2 μM) with human breast adenocarcinoma cells decreased R1 protein expression in a dose-dependent manner. In contrast incubation with a SEQ ID NO:1 sequence with a four base mismatch or an oligonucleotide with the same ratio of ACTG as SEQ ID NO:1 but scrambled did not decrease R1 protein expression (FIG. 9).

### Example 11

### Target-Specific Inhibition of R1 mRNA Expression by SEQ ID NO:1

In order to examine the specificity of inhibition of R1 mRNA by SEQ ID NO:1, northern blot analyses of other cellular RNA levels in A2058 human melanoma cells treated with SEQ ID NO:1 or a scrambled control analogue of SEQ ID NO:1 were carried out.

Methods.

A2058 human melanoma cells, grown to subconfluency (70-80%), were treated with 0.2 μM of phosphorothioate antisense ODNs for 4 hr in the presence of cationic lipid (Lipofectin reagent, final concentration, 5 μg/ml, GIBCO BRL) and Opti-MEM (GIBCO BRL). Cells were washed once with PBS and incubated for 16 hr in α-MEM medium (GIBCO BRL) containing 10% FBS. Total RNA was prepared in TRIzol reagent (GIBCO BRL) and northern blot analysis was performed as previously described (Hurta and Wright, J. Cell. Biochem. 57: 543-56, 1995) with some modifications. RNA prepared from cells treated with lipofectin alone (Control), SEQ ID NO:1 and scrambled control analogue (SEQ ID NO:1 Scr) were subjected to electrophoresis through 1% formaldehyde agarose gels followed by transfer to nylon membrane. The blots were hybridized with ³²P-labeled probes that detect R1 mRNA, 28S rRNA, 18S rRNA, thioredoxin mRNA, μ-actin mRNA, GAPDH mRNA, thioredoxin reductase mRNA, ribosomal protein S9 mRNA, RNase MRP RNA, RNase P RNA and R2 mRNA.

Results.

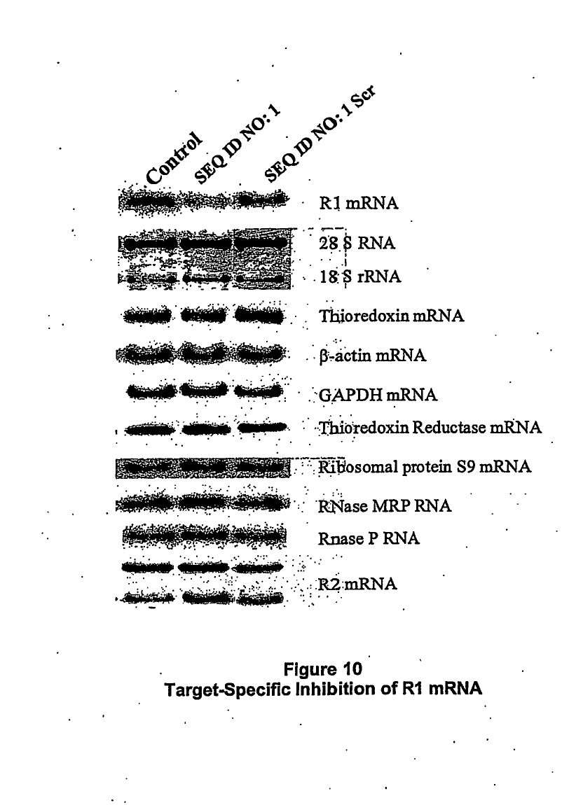

Because no sequence similarities exist between SEQ ID NO:1 target sequence and any of the RNA sequences we selected, SEQ ID NO:1 was not expected to affect the expression of these unrelated cellular RNAs, if SEQ ID NO:1 indeed inhibit R1 mRNA expression target-specifically. As shown in FIG. 10, SEQ ID NO:1 treated cells showed a significant decrease in R1 mRNA but not other RNAs. Furthermore, SEQ ID NO:1 reduced R1 mRNA levels in a highly sequence-specific manner, since no effects were observed on expression of R1 and other cellular RNAs in cells treated with SEQ ID NO:1 scramble control sequence.

SEQ ID NO:1 was found to significantly decrease expression of R1mRNA in a highly target-specific and sequence-specific manner. No effects were observed on expression of other cellular RNAs including 28S rRNA, 18S rRNA, thioredoxin mRNA, β-actin mRNA, GAPDH mRNA, thioredoxin reductase mRNA, ribosomal protein S9 mRNA, RNase MRP RNA, RNase P RNA and R2 mRNA, in cells treated with SEQ ID NO:1 or its scramble control sequence (FIG. 10).

### Example 12

### Phase I Study of SEQ ID NO:1 Given by Continuous Intravenous Infusion (CVI) in Patients with Solid Tumors or Lymphoma

### OBJECTIVES

Primary Objective:

To determine the maximal tolerated dose (MTD) and recommended Phase II dose of SEQ ID NO:1 in patients with solid tumors or lymphoma when administered as a 14-day continuous intravenous infusion.

Secondary Objectives:

To characterize the safety profile of SEQ ID NO:1 when administered to cancer patients as a 14-day continuous infusion. Clinical endpoints, including clinical symptoms, physical examination findings, performance status and adverse events (AEs), will be monitored. Clinical laboratory parameters, including hematology profile, serum chemistry, urinalysis, coagulation and complement split products, will also be monitored.

Tertiary Objective:

To describe the antitumor activity of SEQ ID NO:1 when administered to patients with solid tumors or lymphoma as a 14-day continuous infusion. Antitumor activity will be monitored as a function of level of dose, duration of treatment and type of tumor.

Pharmacokinetic Objective:

To characterize the pharmacokinetic profile of SEQ ID NO:1 when administered to cancer patients as a 14-day continuous infusion.

Study Description:

Eligibility Criteria:

Histologically confirmed diagnosis of solid tumor or lymphoma for which no effective therapy is available or that is unresponsive to conventional therapy
Measurable or evaluable disease (refers to measurability in 1 or more dimensions or a validated tumor marker)
Age≧18 years; Karnofsky performance status of ≧70; informed consent
Able to have a central venous line access maintained throughout the study
No other cancer treatment within 28 days prior to study (within 42 days for nitrosoureas and mitomycin C)
Adequate organ function; PT or aPTT>upper limit of normal
No hematologic malignancy other than lymphoma
No underlying diagnosis or disease state associated with an increased risk of bleeding
No requirement for aspirin, NSAIDS or anticoagulation
No pregnacy, or lactation
No significant infection requiring antibiotic therapy at time of study entry
   Trial Design:
Open-label, single arm, safety and tolerability study
34 patients enrolled on this study
Ascending dose cohorts with a 2-phase dose escalation scheme
First Phase Escalation; cohorts of 1-3 patient; dose doubling until Grade 2 toxicity or dose of 48.0 mg/m²/day (Dose level 4) is completed
Any toxicity equivalent to Grade 2 requires entry of 3 patients and switch to Second Phase Escalation
Second Phase Escalation; At least 3 patients/cohort; dose escalation of 30% until dose limiting toxicity (DLT)
   Treatment Plan:
SEQ ID NO:1 supplied by Lorus Therapeutics Inc. as 100 mg/ml liquid injectable, 5 ml per vial
Treatment cycle 3-weeks duration (14-day continuous infusion and 7-day rest period)
Starting dose of 6.0 mg/m²/day for 14 days followed by a 7 days rest, (cycled twice or more)
Patients received 2 cycles of treatment prior to evaluation of tumor response unless dose-limiting toxicities (DLTs) required removal of patient from the study
Patients permitted to remain on study after the initial 2 cycles, if toxicity remained acceptable and tumor progression has not occurred
Additional response evaluations performed after each additional 2 cycles
   Criteria for Evaluation:
   Efficacy: Tumor response.
   Safety: Adverse events and laboratory evaluation.
   Dosage Selection and Dosing Interval:

The starting dose in the Phase I dose-escalation study is 6.0 mg/m²/day (approximately 0.16 mg/kg/day) infused over 14 days. This dose was selected on the basis of the toxicology data from both the rat and the monkey:
Severe toxicity was noted in Sprague-Dawley rats receiving SEQ ID NO:1 continuously for 14 days at a dose of 10 mg/kg/day (59 mg/m²/day). One-tenth of this dose is 5.9 mg/m²/day.
Minimal and reversible toxicity was noted in the Cynomolgus monkey receiving SEQ ID NO:1 continuously for 14 days at a dose of 10 mg/kg/day (123 mg/m²/day).

In addition, in vivo primary pharmacology studies have demonstrated that SEQ ID NO:1 significantly inhibited the growth of a number of human tumors in mouse models at doses of 1.0 to 10 mg/kg/day. The proposed starting dose of 6 mg/m²/day (0.16 mg/kg/day) in man corresponds to 2 mg/kg/day in the mouse.

Dose Escalation:

Dose escalation schemes and the factors considered to design them are well known in the art and within the purview of the skilled technician. An example of a dose escalation scheme is provided in the Trial Design and in Table 3 for Escalation Phase I and in Table 4 for Escalation Phase II.

TABLE 3
Escalation Phase I (dose doubling): 1 to 3 patients per dose level.

| Dose Level | Dose SEQ ID NO: 1 |
|---|---|
| Dose Level 1 | 6.0 mg/m²/day |
| Dose Level 2 | 12.0 mg/m²/day |
| Dose Level 3 | 24.0 mg/m²/day |
| Dose Level 4 | 48.0 mg/m²/day |

TABLE 4
Escalation Phase II (escalation by 30% increments): minimum 3
patients per dose level.

| Dose Level | Dose SEQ ID NO: 1 |
|---|---|
| Dose Level 5 | 96.0 mg/m²/day |
| Dose Level 6 | 124.8 mg/m²/day |
| Dose Level 7 | 162.2 mg/m²/day |
| Dose Level 8 | 210.9 mg/m²/day |
| Dose Level 9 | 274.2 mg/m²/day |
| Dose Level 10 | 356.5 mg/m²/day |

DLT Definition:
Grade 4 neutropenia associated with fever or lasting 3 days or longer.
Platelet count <25,000 μl.
Any ≧ grade 3 coagulation abnormality (defined by PT and aPTT values).
Clinical hemorrhage ≧ grade 1.
Nausea/vomiting ≧ grade 3 despite maximal antiemetic therapy; diarrhea ≧ grade 3 despite maximal anti-diarrheal therapy.
Any other non-hematological toxicity ≧grade 3 with the exception of alopecia
   Interim Evaluation Status:

Preliminary evaluability assessment was performed. In the Phase I open label single arm study, SEQ ID NO:1 was administered in daily doses escalating from 6.0 mg/m² to 210.9 mg/m² in patients with solid tumors or lymphoma. SEQ ID NO:1 was administered as monotherapy by ambulatory intravenous infusion for 14 days in each 21 day cycle. Interim findings indicate that SEQ ID NO:1 was well tolerated within this dose range. There were no drug related serious adverse events up to and including the 210.9 mg/m² dose. Expected toxicities for agents in the same class as SEQ ID NO:1, phosphorothioate oligonucleotides, include fatigue, prolonged coagulation (PT/aPITI) times and elevated transaminase ALT/AST) levels. Two patients experienced Grade 3 fatigue, three patients experienced Grade 3 increased ALT, one patient experienced Grade 3 increased AST and one patient experienced a prolonged aPTT. Additional sporadic toxicities considered possibly related to study therapy included Grade 3 hypokalemia, Grade 3 increased blood alkaline phosphatase and Grade 3 diarrhea. No maximum tolerated dose (MTD) has been seen at dose cohorts up to and including 210.9 mg/m²/day. The 210.9 mg/m² dose represents a safe high daily-infused dose commensurate with maximal doses commonly studied with other phosphorothioate oligonucleotides.

### Example 13

### Phase I/II Study of SEQ ID NO:1 and Docetaxel Combination Therapy in Patients with Asymptomatic and Symptomatic Progressive Hormone-Refractory Prostate Cancer (HRPC)

Objectives

Primary Objectives:

1) To determine the recommended Phase II dose of SEQ ID NO:1 when given in combination with docetaxel.

2) To establish the efficacy of SEQ ID NO:1 plus docetaxel in patients with asymptomatic and symptomatic hormone refractory prostate cancer with evaluation of:

a) PSA responses
b) Objective tumor responses when there is measurable soft tissue disease
   Secondary Objective:
1) To determine safety of SEQ ID NO:1 in combination with docetaxel
2) To assess the duration of PSA and objective tumor responses
3) To assess time to PSA, and/or pain, and/or objective or clinical disease progression
4) To assess the incidence and duration of pain response
5) To assess the impact of the treatment on patients' Quality of Life (QOL)
6) To obtain follow-up information on patient survival
   Pharmacokinetic Objective:

To characterize the pharmacokinetic profile of SEQ ID NO:1 and docetaxel in patients with asymptomatic and symptomatic HRPC.

Study Description:

Inclusion Criteria:

Histologically confirmed adenocarcinoma of the prostate
Patients must have metastatic prostate adenocarcinoma
Males aged ≧18 years Patients must have received prior hormonal therapy as defined below:
   Castration by orchiectomy or on LHRH agonists with or without
      i) Antiandrogens
      ii) Antiandrogen withdrawal
      iii) Monotherapy with oral estramustine
      iv) Other hormonal agents such as ketoconazole
      If the patient has been treated with LHRH agonists (i.e. without orchiectomy), this therapy should be continued.
Patients should have documented progression defined by either;
   (a) PSA≧5 ng/mL, with or without measurable disease and two consecutive increases in PSA over a reference value, taken at least 1 week apart. It is recognized that PSA fluctuations are such that confirmatory PSA value might be less than the previous value. In these cases, the patient would still be eligible provided the next PSA be greater than the second PSA. or
   b) Progression of measurable disease (see section 11 for definition of measurable disease). For PSA<5 ng/ml, there must be progression of measurable disease.
The patient must have achieved stable analgesia for a minimum of 7 consecutive days prior to study entry. The patient must keep a pain diary for this 7-day period. Stable analgesia will be defined by both:
no increase by more than one point in the daily PPI scores recorded over 7 consecutive days with an identical PPI score for the last two days
and
no variation of the daily analgesic scores (AS) by more than 25% around the mean AS (mean AS=sum of the 7 daily AS divided by 7), i.e., the 7 daily AS should be within the range of values defined below:
the lowest value should be mean AS−25% mean AS.
the highest value should be mean AS+25% mean AS.
   ECOG performance status of 0-2 (Appendix 1).
   If the patient is receiving medical androgen ablation, a castrate level of testosterone (Serum testosterone of <50 ng/mL) must be present.
   Be able to have a central venous line access maintained throughout the study.
   Appropriate organ function defined by the following:
      PT, aPTT<upper limit of normal.
      Hemoglobin≧10.0 g/dL (may be transfused)
      White Blood Cell Count (WBC)≧3.0/μl
      Absolute Neutrophil Count (ANC)≧1500 cells/μl
      Platelet Count≧100,000/μl
      Serum Creatinine≦195 μmol/L or a 24 hour estimated Creatinine Clearance≧50 ml/min
      Serum Bilirubin<upper limit of normal
      AST (SGOT) and ALT (SGPT)<1.5 times upper limit of normal
   Patient must be abstinent, surgically sterile or utilizing a barrier contraceptive method.
   Provide written informed consent prior to the initiation of protocol therapy.
      Exclusion Criteria:
1. Patient has received prior chemotherapy for Prostate Cancer
2. Radiotherapy, biologic therapy, peripheral anti-androgen or any other investigational drugs within 28 days of beginning study treatment with SEQ ID NO:1.
   At least 4 weeks since prior flutamide and megestrol
   At least 6 weeks since prior bicalutamide and nilutamide
   At least 4 weeks since prior hormonal therapy known to decrease PSA levels (including ketoconazole, aminogluthetimide, finasteride)
   At least 4 weeks since prior treatment with estramustine i.e. monotherapy and by oral route and the patient must have recovered from side effects.
3. A malignancy other than adenocarcinoma of the prostate diagnosed within previous 5 years. An exception is given to non-melanoma skin cancer or superficial bladder cancer.
4. An underlying diagnosis or disease state associated with an increased risk of bleeding.
5. Require anticoagulation that increases PT or aPTT above the normal range (deep vein thrombosis or line prophylaxis is allowed).
6. Require treatment with aspirin or aspirin-containing products or anticipate the use of aspirin during the study.
7. Have a significant infection requiring antibiotic therapy at time of study entry.
8. Are unwilling or unable to comply with the requirements set out by the protocol (i.e. patient visits, blood sampling).
9. Are unwilling or unable to provide written informed consent.
10. Presence of another condition that could constitute a contraindication to entry on this protocol.
11. Patients who have received prior antisense therapy.
12. Patients with a history of hypersensitivity reaction to docetaxel or to drugs formulated with polysorbate 80.
   Allowable Concomitant Treatment:

The use of aspirin or aspirin-containing products throughout the study is prohibited. In addition, the use of any NSAIDs within the first cycle (first 21 days) of the administration of study drug is prohibited.

There have been no formal clinical studies to evaluate the drug interactions of docetaxel with other medications. In vitro studies have shown that the metabolism of docetaxel may be modified by the concomitant administration of compounds which induce, inhibit or are metabolized by (and thus may inhibit the enzyme competitively) cytochrome P450-3A such as cyclosporine, terfenadine, ketoconazole, erythromycin and troleandomycin. As a result, caution should be exercised when treating patients with these drugs as concomitant therapy since there is a potential for a significant interaction.

Endocrine Therapy:

Concurrent primary testicular androgen suppression therapy (e.g., with a LHRH analog) allowed.
Steroids as indicated per protocol or at previously prescribed stable (4 weeks prior to study entry) doses are allowed.
Intermittent dexamethasone as an antiemetic is not allowed.

All concomitant medication used during the course of this study will be recorded in the case report forms (CRFs).

Trial Design:

Methodology:

Open-label non-randomized Phase I/II study. Phase I portion will escalate the dose of SEQ ID NO:1 in combination with a fixed dose of docetaxel in order to develop the recommended dose for the Phase II portion. A one-stage design will be utilized for the Phase II portion with a target activity level of 50% and a lower activity level of 20%. A treatment cycle will be 3 weeks duration, 14-day continuous infusion of SEQ ID NO:1 with a 30-60 minute infusion of docetaxel on day 15 followed by a 7-day rest period. Patients will continue treatment for at least 3 cycles unless a patient develops progressive disease or intolerable toxicity. Patients, who respond, have minor responses, or no change in disease status may continue on treatment until disease progression.

Number of Patients:

Approximately 9 to 12 patients will be enrolled in the Phase I portion of this study. Approximately 35 patients (32 evaluable) will be enrolled in the Phase II portion. Phase II sample size will include phase I patients at the phase II dose.

Test Product, Dose and Mode of Administration, Batch No.:

SEQ ID NO:1 will be supplied by Lorus Therapeutics Inc. as 100 mg/ml liquid injectable, 5 ml per vial. Docetaxel is commercially available.

Duration of Treatment and Dosage:

SEQ ID NO: 1 will be administered as a continuous intravenous infusion for 14 days at a starting dose of 124.8 mg/m²/day in combination with docetaxel which will be administered intravenously as a 30-60 minute infusion on day 15 at a fixed dose of 60 mg/m² followed by 7 days of rest.

Criteria for Evaluation:

Efficacy: PSA response, tumor response (by RECIST criteria) if applicable, duration of response, time to progression, incidence and duration of pain response, and Quality of Life.

Safety: Adverse events and laboratory evaluation

Dosage Regimen:

Dose Escalation of Combination Therapy

Escalation Phase I: Patients will be accrued in cohorts of 3 with each patient beginning therapy no sooner than one week apart. The initial dose level will be 124.8 mg/m²/day for SEQ ID NO:1 and 60 mg/m² every 3 weeks for docetaxel (Dose Level 0). Escalation to Dose Level 1 may proceed if no dose-limiting toxicity (DLT) is observed after all patients have completed one treatment cycle (see Table 5). However, if one DLT is observed in the first 3 patients at Dose Level 0, then an additional 3 patients will be treated at Dose Level 0. If less than 2 DLTs are observed in the first 6 patients at Dose Level 0, then accrual at Dose Level 1 may proceed. However, if 2 of 6 patients experience DLTs at Dose Level 0, then Dose Level 0 will be chosen as the dose for Phase II portion of the study.

If >1 DLT in the first 3 patients or >2 DLT in the first 6 patients occur, the doses of docetaxel may be reduced one dose level (Table 5), and the above process will be repeated to establish a Phase II dose level. If no DLTs are seen in the 3 patients at Dose Level 1, then an additional 3 patients will be treated at Dose Level 2. If no DLTs are seen in 3 patients at Dose level 2 then this dose will be used in the Phase II portion of the study. If 1 DLT is seen among 3 patients, the cohort will be expanded to 6 patients. If 2 of 6 patients at Dose Level 1 or 2 experience DLT, then Dose Level below it will be used in the Phase II portion of the study.

If a dose reduction of docetaxel is required, this will not preclude additional cohorts to escalate the SEQ ID NO:1 dose without escalating the docetaxel dose.

TABLE 5
Phase I Dose Escalation

| DOSE LEVEL | DOCETAXEL Q3 WEEKS | SEQ ID NO: 1 |
|---|---|---|
| 2 | 75 mg/m² | 210.9 mg/m²/d |
| 1 | 75 mg/m² | 162.2 mg/m²/d |
| 0* | 60 mg/m² | 124.8 mg/m²/d |
| −1 | 45 mg/m² | 124.8 mg/m²/d |

*Starting dose level

Dose-Limiting Toxicity
Grade 4 neutropenia lasting 3 days or longer, or grade 3/4 neutropenia associated with grade 2 or greater fever.
Any grade 4 thrombocytopenia or grade 3 thrombocytopenia associated with ≧grade 1 hemorrhage.
Any ≧grade 3 coagulation abnormality (defined by PT and aPTT values) associated with clinical hemorrhage ≧grade 1.
Any ≧grade 3 Liver function abnormalities.
Delay of at least 14 days in initiating the second cycle of therapy due to persistent, treatment-related toxicity ≧2.
Any grade 3/4 non-hematologic toxicity except toxicities evaluated as related only to docetaxel.

The occurrence of a DLT does not automatically require a patient to discontinue therapy.

Dose Modification Guidelines for SEQ ID NO:1

In the event of dose limiting toxicities as defined above, SEQ ID NO:1 dose may be reduced one level or interrupted at the investigator's clinical discretion, until the grade reduces to Grade 1 or less. Dosage may then be resumed at the reduced dose level. In the event of unexpected toxicities the same actions may be taken except that resumption of treatment may be at the full dose at the investigator's discretion. Doses should be adjusted to the following recommendations

TABLE 6
Dose levels for SEQ ID NO: 1 dose reductions.
SEQ ID NO: 1

210.9 mg/m²/d
162.2 mg/m²/d
124.8 mg/m²/d

Dose Modification Guidelines for Docetaxel

If toxicity requires a dose to be held (as described below), that dose will be omitted and the next scheduled dose will be delivered as scheduled without delay. A reduced dose will not be re-escalated throughout the remainder of the patient's time on study. The maximum number of dose reductions of docetaxel in a patient is two. If a third dose reduction is needed, the patient will discontinue docetaxel and will stop study treatment. Study treatment will be discontinued if chemotherapy is withheld or interrupted for 4 weeks.

Doses should be adjusted according to the following recommendations (table 7).

TABLE 7
Dose levels for docetaxel dose reductions
DOCETAXEL Q3 WEEKS

75 mg/m²
60 mg/m²
45 mg/m²

### Example 14

### Effects of SEQ ID NO:10N SIHA Cervical Tumor Growth in SCID Mice

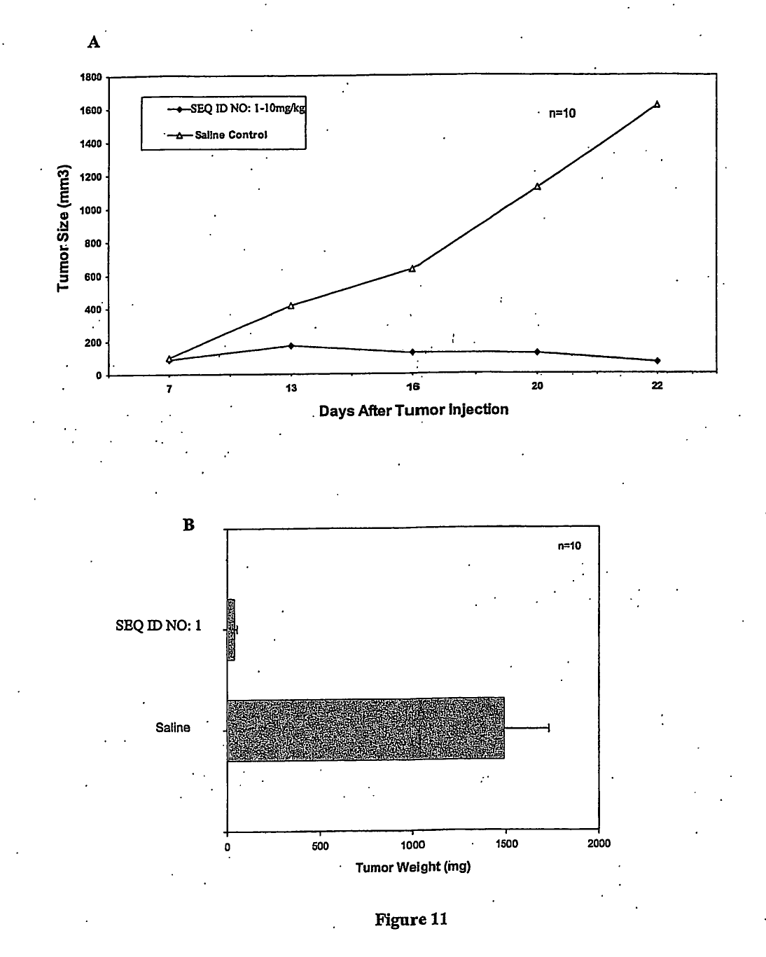

SIHA human cervical cancer cells (1×10⁷ cells in 100 μl of PBS) were subcutaneously injected into the right flank of 6-7 week old female SCID mice. After the size of tumor reached an approximate volume of 100 mm³, 7 days post tumor cell injection, SEQ ID NO:1 was administered by bolus infusion into the tail vein every other day at 10 mg/kg. Control animals received saline alone for the same period. Treatments lasted for 16 days thereafter. Antitumor activities were estimated by the inhibition of tumor volume (FIG. 11A), which was measured with a caliper on five different occasions over 16-day period. Each point represents mean tumor volume calculated from 10 animals per experimental group. As illustrated in FIGS. 11A and 11B, SEQ ID NO:1 treatment demonstrated strong inhibitory effects on the growth of human cervical carcinoma. The bottom panel (FIG. 11B) shows the results of weight measurements of tumors excised from the above animals at the end of the treatments, again demonstrating strong antitumor effect of SEQ ID NO:1.

### Example 15

### Reduction of Lung Nodule Formation by SEQ ID NO:1

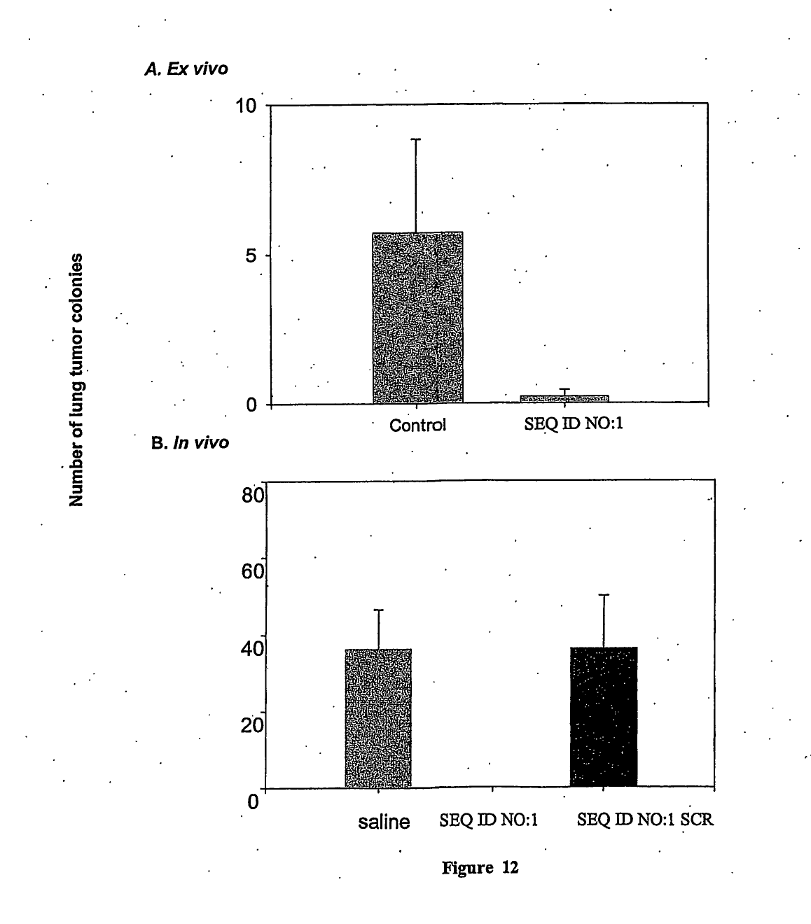

A. Experimental metastasis of C8161 human melanoma cells treated with SEQ ID NO:1 was estimated as follows. Aliquots of C8161 cell suspension were seeded into 100 mm tissue culture dishes at a density of 2×10⁶ and incubated overnight at 37° C. in α-MEM medium supplemented with 10% FBS. Following a wash with 10 ml of PBS, cells were treated for 4 hr with 0.2 μM of SEQ ID NO:1 in the presence of a cationic lipid (Lipofectin reagent, final concentration, 10 μg/ml, Gibco BRL) or with lipofectin alone (CONTROL) as a control. SEQ ID NO:1 was removed by washing the cells once with PBS. Cells were then collected by trypsinization and centrifugation. Approximately 1×10⁵ cells suspended in 0.2 ml of PBS were injected into the tail veins of 6-8 week-old CD-1 athymic female nude mice. Estimates of the number of lung tumors were made 4 weeks later by counting metastatic foci formed on the excised lung surface, see FIG. 12A. Bars represent mean number of tumor nodules in lungs obtained from 7-9 animals per experimental group. Treatment of C8161 cells with SEQ ID NO:1 significantly reduced the formation of lung colonies.

B. C8161 human metastatic melanoma cells were seeded into 100 mm tissue culture dishes at a density of 2×10⁶ and incubated overnight at 37° C. in α-MBM medium supplemented with 10% FBS. The cells were trypsinized, collected by centrifugation and aliquots were removed from the suspension to determine the cell viability using trypan blue exclusion test. Approximately, 1×10⁵ cells suspended in 0.1 ml of PBS were injected into the tail veins of 6-8 week old female SCID mice. Treatment with saline or 10 mg/kg/48 hr SEQ ID NO:1 or SEQ ID NO:1-SCR (scrambled control) was initiated after 2 days. Estimates of the number of lung nodules were made 5-7 weeks later, after excised lungs from individual mice were stained with picric acid dye solution (75% picric acid, 20% formaldehyde, 5% glacial acetic acid), see FIG. 12B. The bars represent the mean number of nodules per mouse with standard error. In the SEQ ID NO:1 treatment group there were no visible lung nodules.

### Example 16

### Effects of Combination Therapy on HT-29 Colon Tumor Growth in Nude Mice

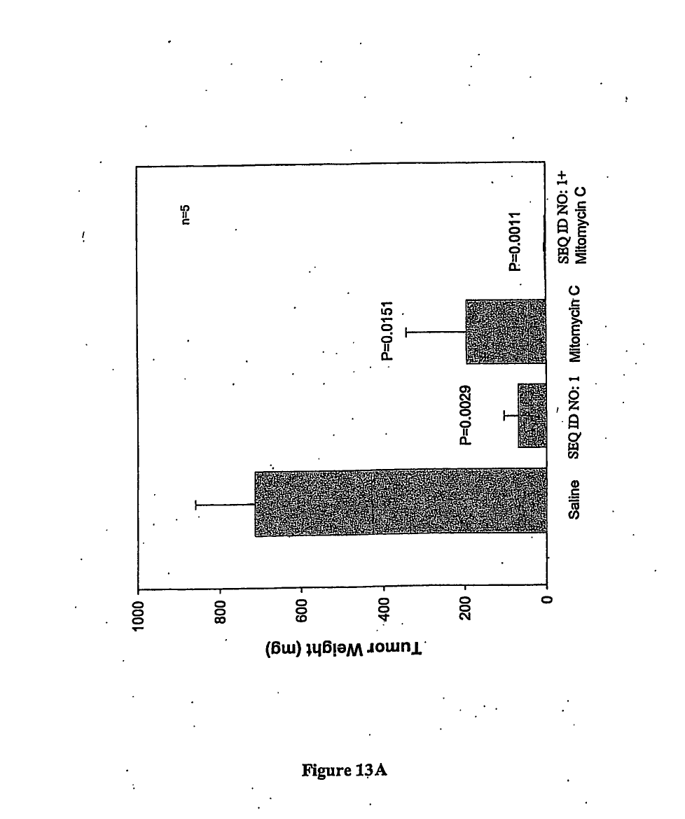

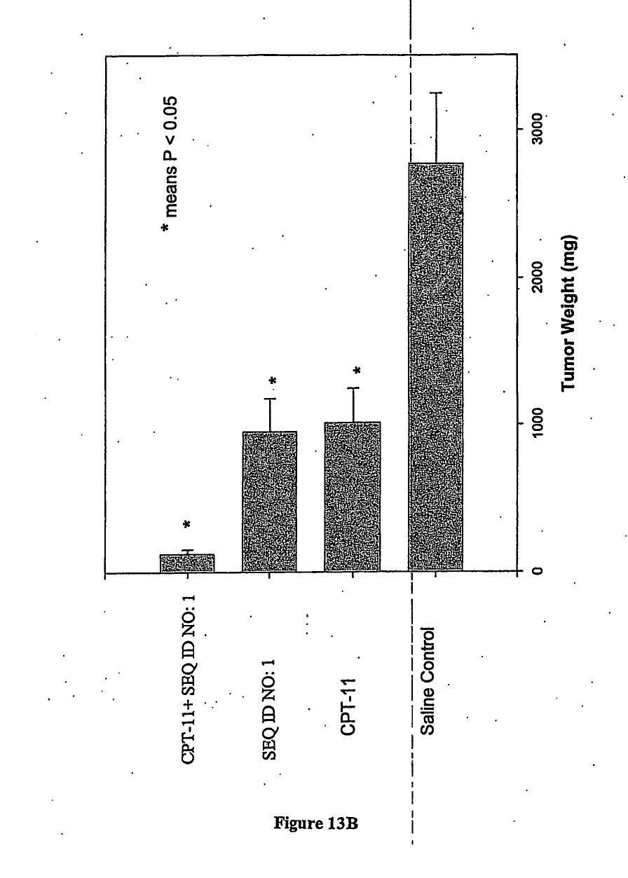

A. HT-29 human colon cancer cells (3×10⁶ cells in 100 μl of PBS) were subcutaneously injected into the right flank of 6-7 weeks old female CD-1 nude mice. After the size of tumor reached an approximate volume of 50 mm³, 4 days post tumor cell injection, SEQ ID NO:1 was administered by bolus infusion into the tail vein every other day at 10 mg/kg. Control animals received saline alone for the same period. Antitumor effect of SEQ ID NO:1 was further compared to that of mitomycin C alone or that of SEQ ID NO:1 in combination with mitomycin C. Mitomycin C was administered intravenously at days 4, 11 and 18 with a dose of 3.5 mg/kg/week, one hour after the treatments with SEQ ID NO:1. All treatments were stopped at day 22. A day after the last treatment, tumors were excised from the animals and their weights were measured (FIG. 13A). A standard bar graph was used to demonstrate the differences in tumor weights with each bar representing mean tumor weight calculated from 5 animals. As illustrated, SEQ ID NO:1 treatments resulted in significant delay of tumor growth compared to saline control. The delay in tumor growth achieved with SEQ. ID NO:1 was superior to the inhibitory effects observed with mitomycin C alone. The combination treatments of SEQ ID NO:1 and mitomycin C showed excellent cooperative effects that are significantly more potent than either agent alone.

B. HT-29 human colon cancer cells (3×10⁶ cells in 100 μl of PBS) were subcutaneously injected into the right flank of 5-6 week old female CD-1 nude mice. After the size of tumor reached an approximate volume of 100 mm³, 7 days post tumor cell injection, SEQ ID NO:1 was administered by bolus infusion into the tail vein every other day at 10 mg/kg. Control animals received saline alone for the same period. Antitumor effect of SEQ ID NO:1 was further compared to that of CPT-11 alone or that of SEQ ID NO:1 in combination with CPT-11. CPT-11 was administered intraperitoneally for 5 days in a row from day 7-12 with a dose of 20 mg/kg in 100 μl saline. All treatments were stopped at day 32. A day after the last treatment, tumors were excised from the animals and their weights were measured (FIG. 13B). A standard bar graph was used to demonstrate the differences in tumor weights with each bar representing mean tumor weight calculated from 9 animals. As illustrated, SEQ ID NO:1 treatments resulted in significant delay of tumor growth compared to saline control. The delay in tumor growth achieved with SEQ ID NO:1 was similar to the inhibitory effects observed with CPT-11 alone. The combination treatments of SEQ ID NO:1 and CPT-11 showed excellent cooperative effects that are significantly more potent than either agent alone.

### Example 17

### Effects of SEQ ID NO:1 in the Treatment of Human Breast Adenocarcinoma Resistant to Cisplatin in SCID Mice

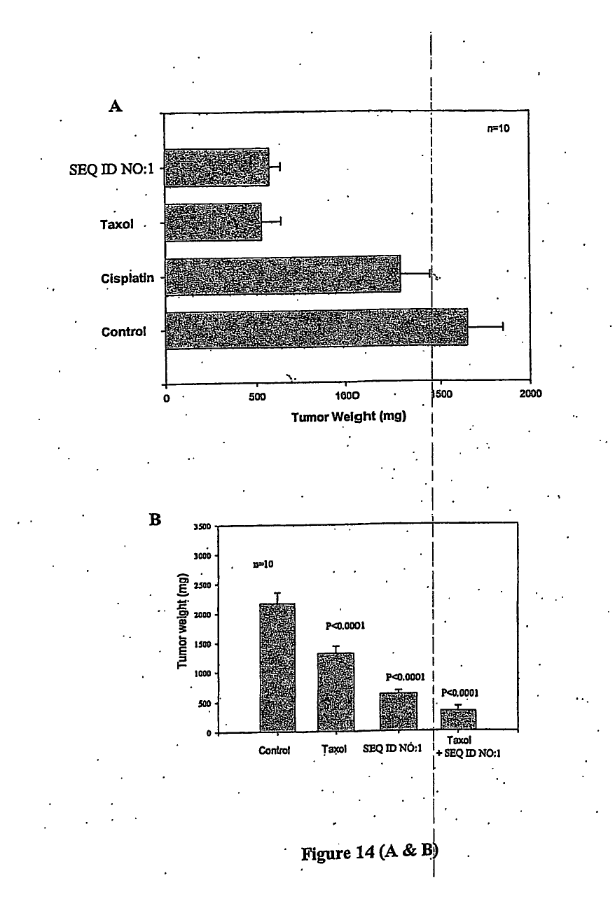

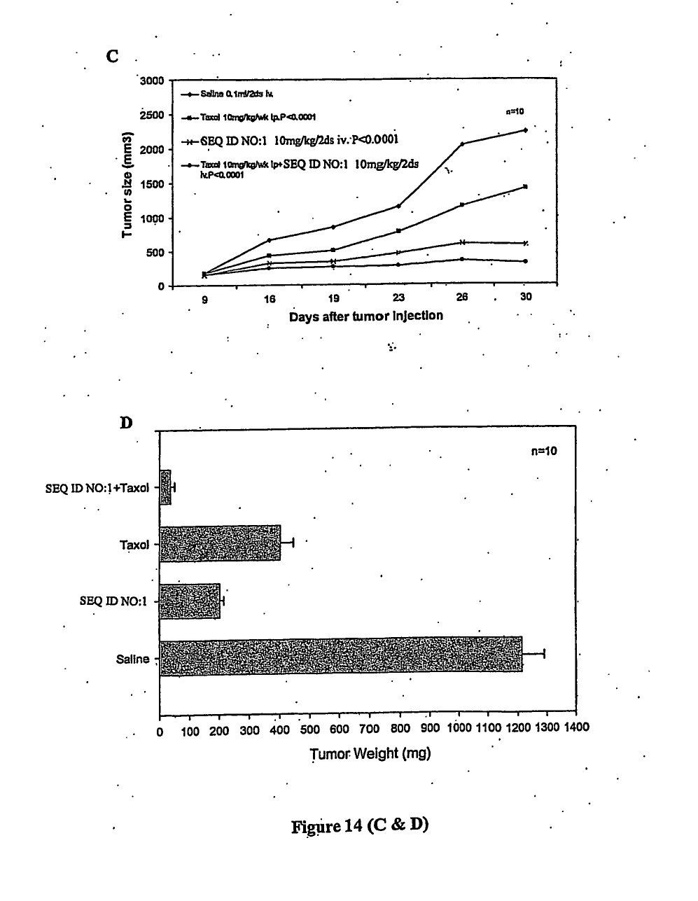

MDA-CDDP-S4 human in vivo-selected Cisplatin-resistant breast adenocarcinoma cells MDA231/CDDPs4) (4×10⁶ cells in 100 μl of PBS) were injected into the fat pad (inside of right leg) of 6-7 weeks old female SCID mice. After the size of tumor reached an approximate volume of 100 mm³, 7-9 days post tumor cell injection, SEQ ID NO:1 was administered by bolus infusion into the tail vein every other day at 10 mg/kg. Control animals received saline alone for the same period. Antitumor effect of SEQ ID NO:1 was further compared to that of Cisplatin or Taxol alone (FIG. 14A) and in combination as indicated in the FIGS. 14B, 14C and 14D. Cisplatin was administered intravenously once a week for three weeks at a dose of 4 mg/kg. Taxol was administered intravenously once a week for three weeks at a dose of 10 mg/kg. Antitumor activities were estimated by the inhibition of tumor volume, which was measured with caliper and each point represents mean tumor volume calculated from 10 animals per experimental group (FIG. 14C). Tumor weight data is presented in the FIGS. 14A, 14C and 14D. At the end of the study animals were sacrificed, tumor weights taken and mean tumor weights are indicated. As illustrated, SEQ ID NO:1 treatments caused significant reduction of tumor weight compared to saline control. As expected, treatment with Cisplatin during the same period was ineffective against Cisplatin-resistant tumor. The delay in tumor growth achieved with SEQ ID NO:1 was superior to the inhibitory effects observed with Taxol, which was used as a positive control. The effects of combined treatment were greater than either treatment alone.

### Example 18

### Effects of SEQ ID NO:1 in the Treatment of Human Breast Adenocarcinoma Resistant to Taxol in SCID Mice

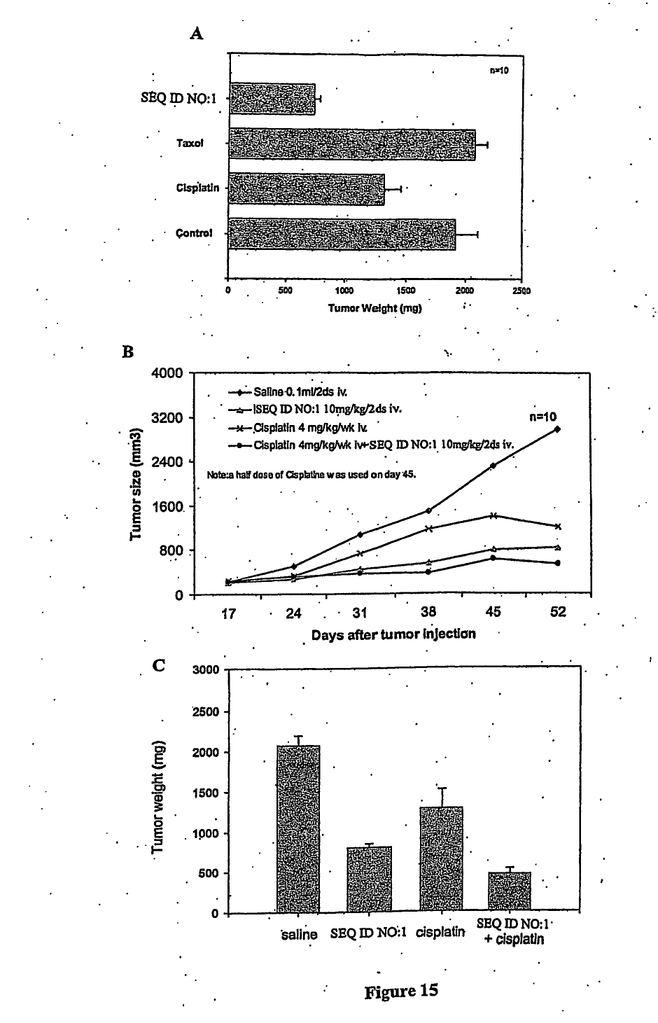

MDA-MB435-To.1 human Taxol-resistant breast adenocarcinoma cells (4×10⁶ cells in 100 μl of PBS) were injected into the fat pad (inside of right leg) of 6-7 weeks old female SCD mice. After the size of tumor reached an approximate volume of 100 mm³, 20 days post tumor cell injection, SEQ ID NO:1 was administered by bolus infusion into the tail vein every other day at 10 mg/kg 15 times. Control animals received saline alone for the same period. Antitumor effect of SEQ ID NO:1 was further compared to that of Cisplatin or Taxol alone (FIG. 15A). Cisplatin was administered intravenously once a week for four weeks at a dose of 4 mg/kg. Taxol was administered intravenously once a week for four weeks at a dose of 20 mg/kg. Antitumor activities were estimated by the inhibition of tumor volume, which was measured with caliper (FIG. 15B). Each point represents mean tumor volume calculated from 9-10 animals per experimental group. As illustrated, SEQ ID NO:1 treatments caused significant reduction of tumor weight compared to saline control. As expected, treatment with Taxol during the same period was ineffective against Taxol-resistant tumor. The delay in tumor growth achieved with SEQ ID NO:1 was superior to the inhibitory effects observed with Cisplatin, which was used as a positive control.

MDA-MB435-To.1 human Taxol-resistant breast adenocarcinoma cells (4×10⁶ cells in 100 μl of PBS) were injected into the fat pad (inside of right leg) of 6-7 weeks old female CB-17 SCID mice. After the size of tumor reached an approximate volume of 100 mm³, 17 days post tumor cell injection, SEQ ID NO:1 was administered by bolus infusion into the tail vein every other day at 10 mg/kg. Control animals received saline alone for the same period. Antitumor effect of SEQ ID NO:1 was compared to that of Cisplatin alone and in combination. Cisplatin was administered intravenously once a week for four weeks at a dose of 4 mg/kg. Antitumor activities were estimated by the inhibition of tumor volume, which was measured with caliper (FIG. 15B). Each point represents mean tumor volume calculated from 10 animals per experimental group. At the end of the study the animals were sacrificed and tumors weighed (FIG. 15C). As illustrated, SEQ ID NO:1 treatment caused significant reduction of tumor weight compared to saline control. The delay in tumor growth achieved with SEQ ID NO:1 was superior to the inhibitory effects observed with Cisplatin, which was used as a positive control. The combination of the two compounds produced anti-tumor efficacy that was superior to either one alone.

### Example 19

### Effects of SEQ ID NO:1 in the Treatment of LD513, Human Multi-Drug Resistant Colon Adenocarcinoma in SCID Mice

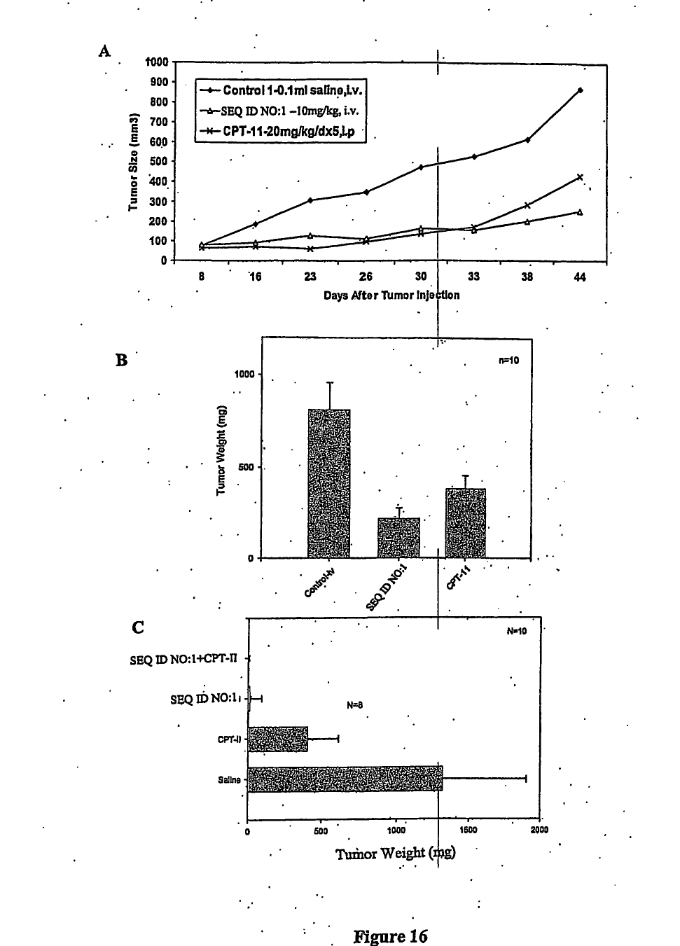

LS513 cells (1×10⁷ cells in 100 μl of PBS) were subcutaneously injected into the right flank of 6-7 weeks old female SCID mice. After the size of tumor reached an approximate volume of 100 mm³, 8 days post tumor cell injection, SEQ ID NO:1 was administered by bolus infusion into the tail vein every other day at 10 mg/kg. Control animals received saline alone for the same period. Antitumor effect of SEQ ID NO:1 was further compared to that of CPT-11. CPT-11 was administered i.p. for 5 days at a dose of 20 mg/kg/day. Antitumor activities were estimated by the inhibition of tumor volume, which was measured with calipers. Each point represents mean tumor volume calculated from 10 animals per experimental group (FIG. 16A). Tumor weights were measured after animals were sacrificed at the end of the treatment (FIGS. 16B and 16C). These cells are not resistant to CPT-11, which was used as a positive control. As illustrated, SEQ ID NO:1 treatment resulted in significant delay of tumor growth compared to saline control. SEQ ID NO:1 is more effective than CPT-11.

### Example 20

### Effects of SEQ ID NO:1 in the Treatment of Human Promyelocytic Leukemia Cells Resistant to Taxol in SCID Mice

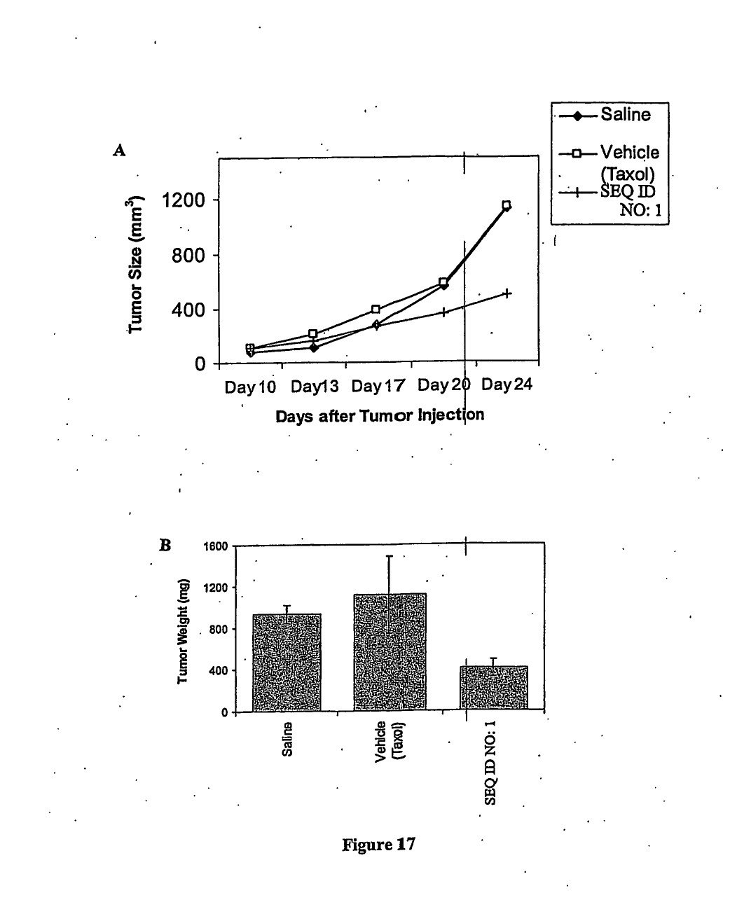

Human taxol-resistant promyelocytic leukemia cells (HL-60) (7×10⁶ cells in 100 μl of PBS) were injected into the right flank of 6-7 weeks old female SCID mice. After the size of tumor reached an approximate volume of 100 mm³, 10 days post tumor cell injection, SEQ ID NO:1 was administered by bolus infusion into the tail vein every other day at 10 mg/kg. Control animals received saline alone for the same period. The anti-tumor effect of SEQ ID NO:1 was further compared to that of taxol. Taxol was administered i.p. once a week at a dose of 10 mg/kg. Anti-tumor activity was estimated by the inhibition of tumor volume, which was measured with caliper (FIG. 17A). Each point represents mean tumor volume calculated from 10 animals per experimental group. In addition animals were sacrificed and tumor weights taken at the end of the study. SEQ ID NO:1 treatment caused significant reduction of tumor weight compared to saline control (FIG. 17B). As expected, treatment with taxol had no effect on tumor growth or weight.

### Example 21

### Prolonged Survival of SCID Mice Bearing Human Burkitt's Lymphoma

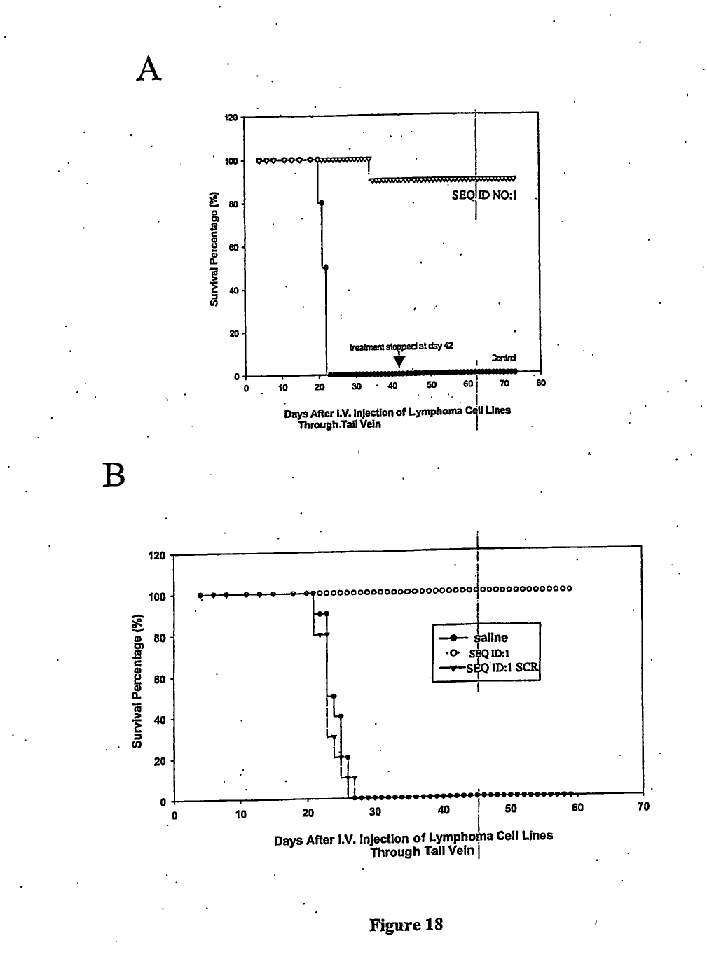

In vivo studies were conducted to assess the therapeutic potential of SEQ ID NO:1 in the treatment of lymphoma. Viable human Burkitt's lymphoma (Raji) cells (5×10⁶) collected from subconfluent logarithmically growing cultures were injected i.v. via the tail vein of each animal and disease was allowed to establish for 2 days. SEQ ID NO:1 in normal saline was administered by tail vein injections every second day at a dose of 10 mg/kg. Control animals received saline alone, without oligonucleotide. Treatment with SEQ ID NO:1 was stopped at day 42. The mice in both groups (n−10) were sacrificed at, day 73. Antitumor effects of SEQ ID NO:1 treatment were assessed by the examination of survival of mice (FIG. 18A). All mice died as a consequence of tumor progression within 23 days when left untreated. All SEQ ID NO:1 treated animals, on the other hand, survived beyond day 73 except one mouse which died at day 35. In an independent experiment, mice survived 140 days, even when treatment was stopped at day 70 (FIG. 18B). This experiment also included treatment with a control oligonucleotide, SEQ ID NO:1-SCR (scrambled version of SEQ ID NO:1). The saline and control ODN mice all died from disease progression by day 27. At day 40, the SEQ ID NO:1-treated mice continued treatment every three days until stopping the treatment at day 59. All mice survived to the end of the experimental period.

### Example 22

### Prolonged Survival of SCID Mice Bearing Mouse Erythroleukemia

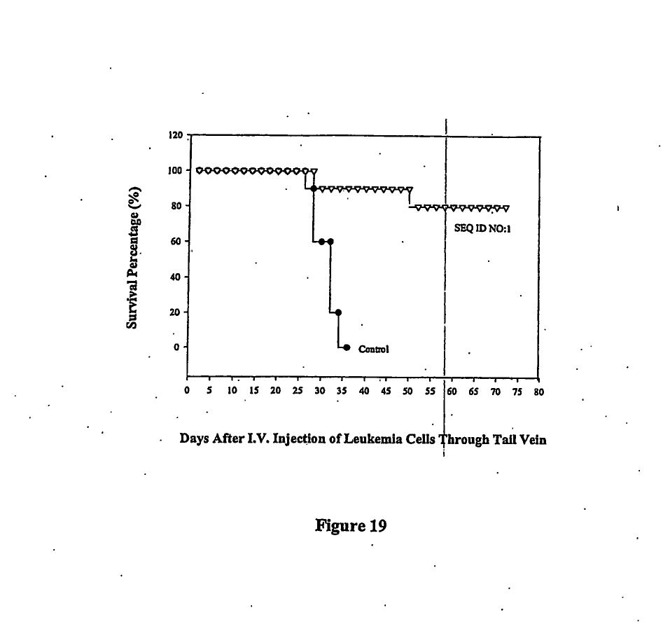

In vivo studies were conducted to assess the therapeutic potential of SEQ ID NO:1 in the treatment of mouse Erythroleukemia. CB7 Friend retrovirus-induced mouse Erytheroleukemia cells (5×10⁶) collected from subconfluent logarithmically growing cultures were injected i.v. via the tail vein of each animal and disease was allowed to establish for 2 days. SEQ ID NO:1 in normal saline was administered by tail vein injections every second day at a dose of 10 mg/kg. Control animals received saline alone, without oligonucleotide. Treatment with SEQ ID NO:1 was stopped at day 71. Antitumor effects of SEQ ID NO:1 treatment were assessed by the examination of survival of mice (FIG. 19). All mice died as a consequence of tumor progression within 36 days when left untreated. All SEQ ID NO:1 treated animals, on the other hand, survived beyond day 71 except one mouse which died at day 30 and another at day 54.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

### REFERENCES

Agrawal S, Temsamani J, Tang J-Y. Pharmacokinetics, biodistribution and stability of oligodeoxynucleotide phosphorothioates in mice. Proc. Natl. Acad: Sci. 1991; 88:7595-7599.
Black L E, Farrelly J G, Cavagnaro J A, Ahn C-H, DeGeorge J J, Taylor A S, et al. Regulatory considerations for oligonucleotide drugs: updated recommendations for pharmacology and toxicology studies. Antisense Research and Development 1994; 4:299-301.
Choy, B. K., McClarty, G. A., Chan, A. K., Thelander, L. and Wright, J. A. (1988). Molecular mechanisms of drug resistance involving ribonucleotide reductase: Hydroxyurea resistance in a series of clonally related mouse cell lines selected in the presence of increasing drug concentrations. Cancer Res. 48: 2029-2035.
Cossum P A, Sasmor H, Dellinger D, Truong L, Cummins L, Owens S R, et al. Disposition of the ¹⁴C-labeled phosphorothioate oligonucleotide ISIS 2105 after intravenous administration to rats. The Journal of Pharmacology and Experimental Therapeutics 1993; 267(3):1181-90.
Cossum P A, Truong L, Owens S R, Markham P M, Shea J P, Crooke S T. Pharmacokinetics of a ¹⁴C-labeled phosphorothioate oligonucleotide, ISIS 2105, after intradermal administration to rats. The Journal of Pharmacology and Experimental Therapeutics 1994; 269(1): 89-94.
Damen, J. E., Gagger, A. Y., Greenberg, A. H. and Wright, J. A. (1989). Generation of metastatic variants in populations of mutator and amplificator mutants. J. Natl. Cancer Inst. 81: 628-631.
DeGeorge J J, Ahn C-H, Andrews P A, Brower M E, Giorgio D W, Goheer M A, et al. Regulatory considerations for preclinical development of anticancer drugs. Cancer Chemother Pharmacol 1998; 41:173-185
Fan, H., Villegas, C., Huang, A. and Wright, J. A. (1996). Suppression of malignancy by the 3′ untranslated regions of ribonucleotide reductase R1 and R2 messenger RNAs. Cancer Res. 56: 4366-4369.
Fan, H., Villegas, C., Huang, A. and Wright, J. A. (1998). The mammalian ribonucleotide reductase R2 component cooperates with a variety of oncogenes in mechanisms of cellular transformation. Cancer Res. 58: 1650-1653.
Geary R S, Leeds J M, Fitchett J, Burcidn T, Truong L, Spainhour C, et al. Pharmacokinetics and metabolism in mice of a phosphorothioate oligonucleotide antisense inhibitor of C-RAF-1 kinase expression. Drug Metabolism and Disposition 1997; 25(11):1272-81.
Gears R S, Leeds J M, Henry S P, Monteith D K, Levin A A. Antisense oligonucleotide inhibitors for the treatment of cancer: 1. Pharmacokinetic properties of phosphorothioate oligodeoxynucleotides. Anti-Cancer Drug Design 1997; 12:383-93.
Grindel J M, Musick T J, Jiang Z, Roskey A, Agrawal S. Pharmacokinetics and metabolism of an oligodeoxynucleotide phosphorothioate (GEM91®) in cynomolgus monkeys following intravenous infusion. Antisense & Nucleic Acid Drug Development 1998; 8:43-52.
Henry S P, Templin M V, Gillett N, Rojko J, Levin A A. Correlation of toxicity and pharmacokinetic properties of a phosphorothioate oligonucleotide designed to inhibit ICAM-1. Toxicologic Pathology 1999; 27(1):95-100.
Ho, P. T. C. and Parkinson, D. R. (1997). Antisense oligonucleotides as therapeutics for malignant disease. Semin. Oncol. 24: 187-202.
Huang, A. and Wright, J. A. (1994). Fibroblast growth factor mediated alterations in drug resistance, and evidence of gene amplification. Oncogene 9: 491-499.
Hurta R. A. and Wright J. A. (1995) Malignant transformation by H-ras results in aberrant regulation of ribonucleotide reductase gene expression by transforming growth factor-beta 1. J. Cell. Biochem. 57: 543-556.
Iversen P. In vivo studies with phosphorothioate oligonucleotides: pharmacolinetics prologue. Anti-Cancer Drug Design 1991; 6:531-8.
Longo, D. L. Neoplastic Disorders in Principles of Internal Medicine (Harrison's) 14^{th} Ed., A. S. Fauci, E. Braunwald, K. J. Isselbacher, J. D. Wilson, J. B. Martin, D. L. Kasper, S. L. Hauser, D. L. Longo (eds.) New York, McGraw-Hill, 1998, chapter 81.
McClarty, G. A., Chan, A. K., Engstrom, Y., Wright, J. A. and Thelander, L. (1987). Elevated expression of M1 and M2 components and drug-induced post-transcriptional modulation of ribonucleotide reductase in a hydroxyurea-resistant mouse cell line. Biochemistry 26: 8004-8011.
Nocentini, G. (1996). Ribonucleotide reductase inhibitors: New strategies for cancer chemotherapy. Crit. Rev. Oncol/Hematol. 22: 89-126.
Srinivasan S K, Tewary H K, Iversen P L. Characterization of binding sites, extent of binding, and drug interactions of oligonucleotides with albumin. Antisense Research and Development 1995 5:131-9.
Zhang, R., Diasio, R. B., Lu, Z., Liu, T., Jiang, Z., Galbraith, W. and Agrawal, S. (1995). Pharmacokinetics and tissue distribution in rats of an oligodeoxynucleotide phosphorothioate (GEM 91®) developed as a therapeutic agent for human immunodeficiency virus type-1. Biochem. Pharmacol. 49: 929-939.

The embodiments of the invention in which an exclusive property or privilege is claimed are defined as follows:

1. An antisense oligonucleotide of between 7 and 100 nucleotides in length comprising at least 7 consecutive nucleotides from SEQ ID NO:1 for use in the treatment of cancer in a mammal in need of such therapy.

2. The antisense oligonucleotide according to claim 1, wherein said cancer is a solid tumour.

3. The antisense oligonucleotide according to claim 1, wherein said cancer is a leukaemia.

4. The antisense oligonucleotide according to claim 2, wherein said solid tumour is drug-resistant.

5. The antisense oligonucleotide according to claim 2, wherein said solid tumour is metastatic.

6. The antisense oligonucleotide according to any one of claims 2, 4 or 5, wherein said solid tumour is a carcinoma.

7. The antisense oligonucleotide according to any one of claims 2, 4 or 5, wherein said solid tumour is a sarcoma.

8. The antisense oligonucleotide according to any one of claims 2, 4 or 5, wherein said solid tumour is a lymphoma.

9. The antisense oligonucleotide according to any one of claims 2, 4 or 5, wherein said solid tumour is an ovarian tumour, a renal tumour, a cervical tumor or a brain tumour.

10. The antisense oligonucleotide according to claim 7, wherein said sarcoma is fibrosarcoma.

11. The antisense oligonucleotide according to claim 7, wherein said lymphoma is a non-Hodgkin's lymphoma.

12. The antisense oligonucleotide according to claim 3, wherein said leukaemia is acute myeloid leukaemia or chronic myeloid leukaemia.

13. An antisense oligonucleotide of between 7 and 100 nucleotides in length comprising at least 7 consecutive nucleotides from SEQ ID NO:1 for use in combination with one or more chemotherapeutic agents in the treatment of cancer in a mammal in need of such therapy.

14. The antisense oligonucleotide according to claim 13, wherein said cancer is a solid tumour.

15. The antisense oligonucleotide according to claim 13, wherein said cancer is a leukaemia.

16. The antisense oligonucleotide according to claim 14, wherein said solid tumour is drug-resistant.

17. The antisense oligonucleotide according to claim 14, wherein said solid tumour is metastatic.

18. The antisense oligonucleotide according to any one of claims 14, 16 or 17, wherein said solid tumour is a carcinoma.

19. The antisense oligonucleotide according to any one of claims 14, 16 or 17, wherein said solid tumour is a sarcoma.

20. The antisense oligonucleotide according to any one of claims 14, 16 or 17, wherein said solid tumour is a lymphoma.

21. The antisense oligonucleotide according to any one of claims 14, 16, 17 or 18, wherein said solid tumour is selected from the group of: renal tumour, breast tumour, lung tumour, prostate tumour, colon tumour, melanoma, ovarian tumour, cervical tumour, brain tumour, liver tumour, colorectal tumour, pancreatic tumour, genitourinary tumour, gall bladder tumour, head and neck tumour, oesophageal tumour and biliary duct tumour.

22. The antisense oligonucleotide according to any one of claims 14, 16, 17 or 18, wherein said solid tumour is selected from the group of: renal tumour, breast tumour, lung tumour, prostate tumour, colon tumour, melanoma, ovarian tumour, cervical tumour, brain tumour and liver tumour.

23. The antisense oligonucleotide according to any one of claims 14, 16, 17 or 18, wherein said solid tumour is selected from the group of: solid tumours, renal tumour, breast tumour, cervical tumor, lung tumour, prostate tumour and colon tumour.

24. The antisense oligonucleotide according to claim 15, wherein said leukaemia is acute myeloid leukaemia, acute promyelocytic leukemia or chronic myeloid leukaemia.

25. The antisense oligonucleotide according to claim 15, wherein said leukaemia is acute myeloid leukaemia.

26. The antisense oligonucleotide according to any one of claims 13 to 25, wherein said one or more chemotherapeutic agents is selected from the group of: capecitabine, 5-fluorouracil, vinblastine, cytarabine, taxol, docetaxel, mitoxantrone, oxaliplatin, mitomycin, irinotecan, dacarbazine, cisplatin, hydroxyurea, gemcitabine, prednisone, idarubicin, etoposide, fludarabine, filgrastin, carboplatin, mitomycin C, paclitaxel and interleukin-2 or a combination thereof.

27. The antisense oligonucleotide according to any one of claims 13 to 25, wherein said one or more chemotherapeutic agents is selected from the group of: capecitabine, 5-fluorouracil, cytarabine, taxol, docetaxel, mitoxantrone, oxaliplatin, mitomycin, irinotecan, dacarbazine, cisplatin and gemcitabine, or a combination thereof.

28. The antisense oligonucleotide according to any one of claims 13 to 25, wherein said one or more chemotherapeutic agents is selected from the group of: capecitabine, cytarabine, taxol, docetaxel, oxaliplatin and gemcitabine, or a combination thereof.

29. The antisense oligonucleotide according to any one of claims 1 to 28, wherein said antisense oligonucleotide comprises a sequence as set forth in SEQ ID NO:1.

30. The antisense oligonucleotide according to any one of claims 1 to 28, wherein said antisense oligonucleotide consists of a sequence as set forth in SEQ ID NO:1.

31. The antisense oligonucleotide according to any one of claims 1 to 30, wherein said antisense oligonucleotide comprises one or more phosphorothioate internucleotide linkages.

32. The antisense oligonucleotide according to any one of claims 1 to 31, wherein said mammal is a human

33. An antisense oligonucleotide of between 20 and 100 nucleotides in length comprising the sequence as set forth in SEQ ID NO:1 for use in combination with one or more chemotherapeutic agents in the treatment of a human having a cancer selected from the group of: a solid tumour, lymphoma, renal cancer, breast cancer, lung cancer, prostate cancer, ovarian cancer, cervical cancer, colon cancer and leukaemia.

34. The antisense oligonucleotide according to claim 33, wherein said antisense oligonucleotide consists of a sequence as set forth in SEQ ID NO:1.

35. The antisense oligonucleotide according to claim 33 or 34, wherein said antisense oligonucleotide comprises one or more phosphorothioate internucleotide linkages.

36. The antisense oligonucleotide according to any one of claims 33 to 35, wherein said one or more chemotherapeutic agent is gemcitabine and said cancer is a solid tumour.

37. The antisense oligonucleotide according to any one of claims 33 to 35, wherein said one or more chemotherapeutic agent is irinotecan or mitomycin C and said cancer is colon cancer.

38. The antisense oligonucleotide according to any one of claims 33 to 35, wherein said one or more chemotherapeutic agent is paclitaxel or cisplatin and said cancer is breast cancer.

39. The antisense oligonucleotide according to any one of claims 33 to 35, wherein said one or more chemotherapeutic agent is docetaxel and said cancer is prostate cancer.

40. The antisense oligonucleotide according to any one of claims 33 to 35, wherein said one or more chemotherapeutic agent is a combination of oxaliplatin and capecitabine and said cancer is colon cancer.

41. The antisense oligonucleotide according to any one of claims 33 to 35, wherein said one or more chemotherapeutic agent is cytarabine and said cancer is acute myeloid leukaemia.

42. The antisense oligonucleotide according to claim 37, wherein said renal cancer is advanced renal cancer.

43. The antisense oligonucleotide according to claim 37, wherein said renal cancer is metastatic renal cancer.

44. The antisense oligonucleotide according to any one of claims 37, 42 or 43, wherein said antisense oligonucleotide is formulated for administration to said mammal at a dose of between about 124.8 mg/m²/day and about 274.2 mg/m²/day.

45. Use of an antisense oligonucleotide of between 7 and 100 nucleotides in length comprising at least 7 consecutive nucleotides from SEQ ID NO:1 in the manufacture of a medicament for the treatment of cancer.

46. The use according to claim 45, wherein said cancer is a solid tumour.

47. The use according to claim 45, wherein said cancer is a leukaemia.

48. The use according to claim 46, wherein said solid tumour is drug-resistant.

49. The use according to claim 46, wherein said solid tumour is metastatic.

50. The use according to any one of claims 46, 48 or 49, wherein said solid tumour is a carcinoma.

51. The use according to any one of claims 46, 48 or 49, wherein said solid tumour is a sarcoma.

52. The use according to any one of claims 46, 48 or 49, wherein said solid tumour is a lymphoma.

53. The use according to any one of claims 46, 48 or 49, wherein said solid tumour is an ovarian tumour, a renal tumour or a brain tumour.

54. The use according to claim 51, wherein said sarcoma is fibrosarcoma.

55. The use according to claim 52, wherein said lymphoma is a non-Hodgkin's lymphoma.

56. The use according to claim 47, wherein said leukaemia is acute myeloid leukaemia or chronic myeloid leukaemia.
